(19) United States
(12) Patent Application Publication
(10) Pub. No.: US 2023/0103369 A1
(43) Pub. Date: Apr. 6, 2023

(54) THE USE OF MOLECULARLY IMPRINTED POLYMERS FOR THE RAPID DETECTION OF EMERGING VIRAL OUTBREAKS

(71) Applicant: 6TH WAVE INNOVATIONS CORP, Baltimore, MD (US)

(72) Inventors: Jonathan P. GLUCKMAN, Timberlea, CA (US); Sherman G. MCGILL, Munford, TN (US); Aristotle G. KALIVRETENOS, Ellicott City, MD (US); Guneet KUMAR, Ellicott City, MD (US); Louis W. REICHEL, Ellicott City, MD (US); Brandi MAULL, Salt Lake City, UT (US); Garrett KRAFT, Cottonwood Heights, UT (US); Dae Jung KIM, Herriman, UT (US)

(21) Appl. No.: 17/914,755

(22) PCT Filed: Mar. 29, 2021
(86) PCT No.: PCT/US2021/024670

§ 371 Date: Sep. 26, 2022

Related U.S. Application Data
(60) Provisional application No. 63/000,977, filed on Mar. 27, 2020.
(60) Provisional application No. 63/068,007, filed on Aug. 20, 2020.
(60) Provisional application No. 63/010,244, filed on Apr. 15, 2020.

(51) Int. Cl. G01N 33/569; G01N 29/12; G01N 29/24; G01N 33/543

(52) U.S. Cl. CPC G01N 33/56983; G01N 29/12; G01N 29/2443; G01N 33/54366; G01N 2291/014; G01N 2291/023

(57) ABSTRACT

The present disclosure provides molecularly imprinted polymers (MIPs) for selectively binding one or more viruses of a target viral genus, for example SARS-CoV-2, as well as methods of manufacture and uses thereof.

### CROSS-REFERENCE TO RELATED APPLICATIONS

This application claims the benefit of priority to U.S. Application Ser. No. 63/068,007, filed Aug. 20, 2020, U.S. Application Ser. No. 63/010,244, filed, Apr. 15, 2020 and U.S. application Ser. No. 63/000,977, filed Mar. 27, 2020, the contents of each of which are hereby incorporated by reference in their entireties for all purposes.

### BACKGROUND

Viral pandemics are large-scale outbreaks of viral infections that can greatly increase morbidity and mortality over a wide geographic area and cause significant economic, social, and political disruption. The occurrence and severity of viral outbreaks has only increased over the past century as the world has become more interconnected with global travel, increased population density because of urbanization, and changes in land use. Examples of recent viral outbreaks include coronavirus (COVID 2019, MERS 2012, and SARS 2003), influenza viruses (Bird flu H7N9 2013, and Swine flu H1N1 2009), and filoviruses (Zika 2015 and Ebola 2013).

Unlike bacterial based infections, which have a range of antibiotic based treatment options, new viral infections have limited treatment options requiring prevention and containment using quarantines and vaccination to keep the populous safe.

There is an urgent need for methods of rapidly detecting emerging viral outbreaks in order to prevent and contain worldwide pandemics. Early intervention and investment into medical diagnostics is crucial for early detection and containment of new viruses and preservation of the world economy.

### SUMMARY OF THE INVENTION

The present disclosure relates generally to molecularly imprinted polymers and methods of making molecularly imprinted polymers and uses thereof. More particularly, the present disclosure provides materials comprising a molecularly imprinted polymer (MIP) for the efficient, commercially viable and rapid detection of emerging viral outbreaks.

The present disclosure provides, in part, materials comprising a molecularly imprinted polymer which selectively binds one or more viruses of a target viral genus, utilizing in various embodiments, an attenuated or inactivated strain of the target virus(es) or a surrogate for the target virus(es) having a size, shape and morphology substantially similar to all or part of the target virus(es). In some embodiments, the present disclosure relates to molecularly imprinted polymers in the form of beads or molecularly imprinted polymer films having a plurality of complexing cavities which selectively binds one or more viruses of a target viral genus present in a sample (e.g., biological samples, food matrices, environmental samples etc.). Materials comprising a molecularly imprinted polymer of the present disclosure (e.g., macroreticular MIP beads, MIP films, and/or MIP nanoparticles) is/are designed to have a high affinity for all, or part of a target virus, and/or other biomarkers associated with the target virus. Also disclosed herein, are MIP arrays comprising two or more MIPs, wherein each MIP comprises a crosslinked copolymer having a plurality of complexing cavities which selectively binds a target virus.

This disclosure further addresses the need for new MIP technologies (including MIP materials, methods of manufacturing, and methods of using such MIP materials) that can be used to selectively bind, isolate and/or label the target virus(es), with high efficiency for removing the target virus particles, good capacity for the target virus particles, and which are regenerable if the requirements of the particular application so demand.

In one aspect, the present disclosure provides methods, articles, and devices for selectively sequestering one or more viruses of a target viral genus present in a sample with minimal processing. For example, the present disclosure provides methods and devices for selectively sequestering the virus that causes COVID-19 (i.e., SARS-CoV-2). In addition, the present disclosure provides sensors coated with a MIP or MIP array of the present disclosure for detecting a target virus. Also disclosed herein are methods of diagnosing a subject infected with a target virus utilizing MIPs of the present disclosure, in addition to diagnostic kits. The present disclosure also provides, in part, methods for detecting the presence of a target virus on a target area, utilizing MIPs or MIP arrays of the present disclosure. Exemplary target areas include environmental surfaces such as hospitals, airports, medical devices, floor sweepings, bed railings, tables, bedding, cloths, door knobs, and the like.

In addition, the present disclosure provides for methodologies for making high surface area MIPs or MIP arrays to allow for both high selectivity and high capacity for the target virus particles. This is particularly important for sequestration and or removal of large quantities of target viral particles. Further, this methodology allows for production using suspension polymerization methods that yields a product with qualities (hardness, stability, pH tolerance, etc) that allow for use across a broad spectrum of applications and process conditions.

The present disclosure provides a molecularly imprinted polymer (MIP) comprising a crosslinked copolymer having a plurality of complexing cavities which selectively binds one or more viruses of a target viral genus, wherein the copolymer is prepared by polymerizing a mixture comprising:

(a) one or more binding monomers complexed to a template having a size, shape and morphology substantially similar to the viruses of the target viral genus;
(b) optionally one or more non-crosslinking monomers; and
(c) one or more crosslinking monomers;
then removing the template from the copolymer, thereby providing said plurality of complexing cavities (thereby activating the MIP).

In some embodiments, provided herein is a MIP array comprising two or more MIPs, wherein each MIP comprises a crosslinked copolymer having a plurality of complexing cavities which selectively binds a target virus, wherein the target virus of each MIP is different.

In some embodiments, the present disclosure provides an article comprising a MIP comprising a crosslinked copolymer having a plurality of complexing cavities which selectively bind a target virus and a labeling composition comprising a label, wherein the label binds to any target virus bound to the MIP,

wherein said MIP is prepared by polymerizing a mixture comprising:

(a) one or more binding monomers complexed to the target virus; and

(b) optionally one or more non-crosslinking monomers; and

(c) one or more crosslinking monomers;

then removing the virus from the copolymer, thereby providing said plurality of complexing cavities.

In some embodiments, the present disclosure provides a method of detecting a target virus or a target viral genus comprising:
(a) contacting a sample with a MIP of the present disclosure, thereby binding at least a portion of any viruses of the target viral genus present in the sample to the MIP; and
(b) reacting a label molecule capable of attaching to the bound viruses;
wherein the label molecule can be detected to show the presence of the bound virus.

In some embodiments, the present disclosure provides a method of detecting a target virus or a target viral genus comprising:

(a) contacting a sample with an article of the present disclosure, thereby binding any of the target viruses in the sample to one or more of the MIPs; and

(b) reacting a label molecule capable of attaching to the bound viruses;

wherein the label molecule can be detected to show the presence of the bound target virus.

In some embodiments, the molecularly imprinted polymer is coated on a device or component of a device capable of detecting the presence of a virus, such as a quartz crystal microbalance resonator.

In some embodiments, the present disclosure provides a sensor for detecting a virus, coated with a MIP or MIP array of the present disclosure.

In some embodiments, provided herein is a method of removing one or more target viruses from a fluid, comprising contacting the fluid with a MIP or MIP array of the present disclosure whereby at least a portion of any target virus in the fluid binds to the MIP or MIP array.

Unless otherwise defined, all technical and scientific terms used herein have the same meaning as commonly understood by one of ordinary skill in the art to which this disclosure belongs. In the case of conflict, the present specification, including definitions, will control. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents, and other references mentioned herein are incorporated by reference herein, for all purposes. The references cited herein are not admitted to be prior art to the claimed inventions. In addition, the materials, methods, and examples are illustrative only and are not intended to be limiting.

Other features and advantages of the present disclosure will be apparent from the following detailed description and claims.

### BRIEF DESCRIPTION OF THE DRAWINGS

FIG. 1A shows a schematic diagram of the novel coronavirus SARS-CoV-2.

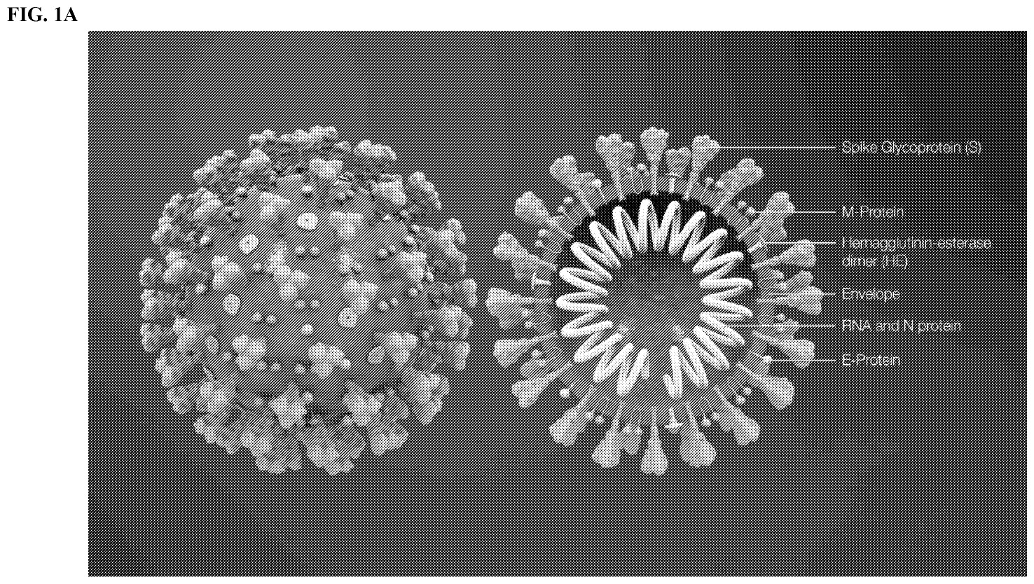

FIG. 1B shows the structure of SARS-CoV-2 S in the prefusion conformation. Side and top views of the prefusion structure of the SARS-CoV-2 S protein with a single RBD in the up conformation. The two RBD down protomers are shown as cryo-EM density in either white or gray and the RBD up protomer is shown in colored ribbons.

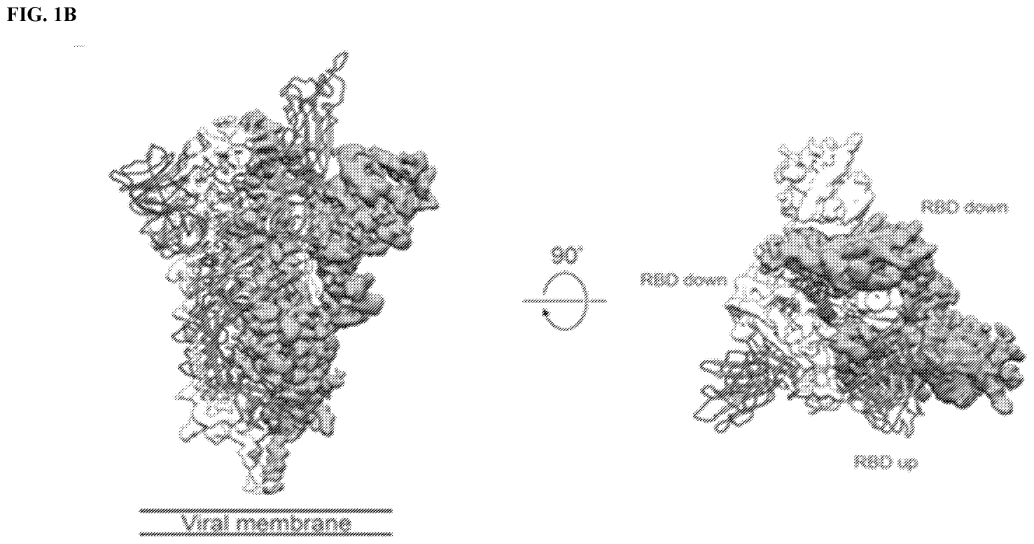

FIG. 2 shows a schematic diagram of the basic components of viral molecularly imprinted polymers.

FIG. 3 shows a schematic diagram a rapid colorimetric test for SARS-CoV-2.

FIG. 4 shows a schematic diagram of a quartz crystal microbalance (QCM) sensor.

FIG. 5 shows a schematic diagram of a lateral flow dipstick.

FIG. 6 shows a schematic diagram of an electrochemical biosensor. To enhance the detection function, the surface of the working electrode is modified with MIPs.

FIG. 7 is a schematic diagram of a typical imprinting process.

FIG. 8 shows atomic force microscopy (AFM) images of virus particles deposited on a glass substrate to create a virus template.

FIG. 9 a) shows an atomic force microscopy (AFM) cantilever positioned over the thin films with a razor blade slice to obtain the height profile and b) an AFM image of the polymer thin film surface topography and graph showing MIP thickness profile.

FIG. 10 shows a) a size distribution graph of imprinting cavities and b) an AFM image of imprinting cavities in the MIP thin film from the virus imprinting process.

FIG. 11 shows QCM adsorption data for QCM MIP thin film sensors (AMIP 26) and non-imprinted controls (NIP3).

FIG. 12 shows QCM-D adsorption data for QSense QCM-D MIP thin film sensors sensors and non-imprinted controls (NIP3).

FIG. 13 shows an images of a) infected cell culture supernatant treated (left image), and b) uninfected cell culture supernatant treated (right image). Both samples underwent the labeling procedure with NHS-Alexa 488 fluorescent dye. Images taken at 20× magnification with the same laser power.

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

The term “about” when immediately preceding a numerical value means a range (e.g., plus or minus 10% of that value). For example, “about 50” can mean 45 to 55, “about 25,000” can mean 22.500 to 27.500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

“Optional” or “optionally” means that the subsequently described element, event or circumstances may or may not occur, and that the description includes instances where said event, element, or circumstance occurs and instances in which it does not.

As used herein, “subject” include vertebrates, more specifically a mammal (e.g., a human, horse, pig, rabbit, dog, sheep, goat, non-human primate, cow, cat, guinea pig or rodent), a fish, a bird or a reptile or an amphibian. In some embodiments, the subject is a human subject. Unless otherwise specified, the term subject does not denote a particular age or sex. In some embodiments, the subject is a geriatric subject, a pediatric subject, a teenage subject, a young adult subject, or a middle aged subject. In some embodiments, the subject is less than about 18 years of age. In some embodiments, the subject is at least about 18 years of age. In some embodiments, the subject is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age. In some embodiments the subject is a male subject. In some embodiments the subject is a female subject. In some embodiments, the subject is diagnosed with or is considered at risk of having or developing a viral infection. In some embodiments, the subject has been diagnosed with or is considered at risk of having or developing COVID-19 (i.e., the disease associated with SARS-CoV-2 infection). The terms “subject” and “patient” are used interchangeably throughout the present application.

In embodiments, a patient population refers to a particular, defined set of subjects having a disease or disorder or at risk of developing a particular disease or disorder.

As used herein, “sample” includes any sample containing or potentially containing a target virus. Samples contemplated herein include, but are not limited to, aerosols, fluid samples, solid samples, biological samples, food matrices, insects, and environmental samples containing or potentially containing a target virus etc. In some embodiments, the sample is droplets or fomites generated by, exhaled breath, coughing and/or sneezing which contain or potentially contain a target virus. In some embodiments, the sample is from environmental surfaces such as hospitals, airports, medical devices, floor sweepings, bed railings, tables, bedding, cloths, door knobs, and the like. In some embodiments, the sample is from a plant which contains or potentially contains a target virus, e.g., a sample from a seed, stem, leaf, mushroom, and the like.

Biological samples, contemplated herein, include, for example, one or more biological samples selected from the group consisting of bioaerosols, biological fluids, tissue extracts and tissues. In some embodiments, the biological fluid can be selected from the group consisting of blood, cerebrospinal fluid, serum, plasma, urine, nipple aspirate, fine needle aspirate, tissue lavage, saliva, sputum, ascites fluid, semen, lymph node sample, vaginal pool, synovial fluid, spinal fluid, amniotic fluid, breast milk, pulmonary sputum or surfactant, urine, fecal matter, fluids collected from any of liver, kidney, breast, bone, bone marrow, testes, brain, ovary, skin, lung, prostate, thyroid, pancreas, cervix, stomach, intestine, colorectal, bladder, colon, nares and uterine, head and neck, nasopharynx tumors, and other liquid samples of biologic origin. In some embodiments, the tissues can be selected from the group consisting of liver, kidney, breast, testes, brain, ovary, skin, head and neck, lung, prostate, thyroid, pancreas, cervix, stomach, intestine, colorectal, bladder, colon, uterine, and other tissue of interest. In one embodiment of the invention, the tissue extracts can be selected from the group consisting of liver, kidney, breast, testes, brain, ovary, skin, head and neck, lung, prostate, thyroid, pancreas, cervix, stomach, intestine, colorectal, bladder, colon, uterine extracts and other tissue extracts of interest. In some embodiments, the bioaerosol is droplets or fomites generated by, e.g., exhaled breath, coughing and/or sneezing.

As used herein, the term “epitope” includes a protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics.

The term “polynucleotide” as referred to herein means a polymeric nucleotide of at least 10 bases in length, either ribonucleotides or deoxynucleotides or a modified form of either type of nucleotide. The term includes single and double stranded forms of DNA.

As used herein, the terms “label” or “labeled” refers to incorporation of a detectable group. The detectable group may be any material having a detectable physical or chemical property, for example detectable by spectroscopic, photochemical, biochemical, immunochemical, fluorescent, electrical, optical or chemical means, for example, magnetic beads (e.g. DYNABEADS®); biotin, avidin, or streptavidin; radioisotopes or radionuclides (e.g., 3H, 14C, 15N, 35S, 90T, 99Tc, 111In, 125I, 131I); fluorescent labels (e.g., Texas-Red, green fluorescent protein, yellow fluorescent protein, cyan fluorescent protein, Alexa dye molecules, FITC, fluorescin and its derivatives, rhodamine and its derivatives, lanthanide phosphors, dansyl, umbelliferone etc); enzymatic labels, which may primarily be hydrolases, particularly phosphatases, esterases and glycosidases, or oxitranscription factoreductases, particularly peroxidases (e.g., alkaline phosphatase, horseradish peroxidase, beta-galactosidase, beta-lactamase, galactose oxidase, lactoperoxidase, luciferase, myeloperoxidase, and amylase); colorimetric labels such as colloidal gold colored glass or plastic (e.g. polystyrene, polypropylene, latex, etc.) beads, and nanoparticles (e.g., gold nanoparticles); chemiluminescent (e.g., luciferin, and 2,3-dihydrophthalazinediones, e.g., luminol); predetermined polypeptide epitopes recognized by a secondary reporter (e.g., leucine zipper pair sequences, binding sites for secondary antibodies, metal binding domains, epitope tags).

The labels provided herein may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions. Labels are often attached by indirect means. Generally, a ligand molecule (e.g., biotin) is covalently bound to the target. The ligand then binds to an anti-ligand (e.g., streptavidin) molecule which is either inherently detectable or covalently bound to a signal system, such as a detectable enzyme, a fluorescent compound, or a chemiluminescent compound. Alternatively, any haptenic or antigenic compound may be used in combination with an antibody. Components may also be conjugated directly to signal-generating compounds, e.g., by conjugation with an enzyme or fluorophore. In some embodiments, the labels provided herein may be attached by spacer arms of various lengths to reduce potential steric hindrance.

As used herein “independent molecular recognition and detection” is defined as a molecular recognition and binding event that is independent of the detection event. Independent recognition and detection processes generally require a rinsing step for accurate detection (e.g., binding>rinse>label molecule>rinse>detection).

As used herein, “coupled molecular recognition and specific detection” is defined as as molecular recognition and binding event that are coupled (e.g., binding>detection).

Throughout the present disclosure, materials comprising molecularly imprinted polymers, methods of making molecularly imprinted polymers and uses thereof are generally expressed as selectively binding one or more viruses of a target viral genus. However, the present disclosure also contemplates embodiments wherein materials comprising molecularly imprinted polymers selectively bind a portion of a target virus, and/or biomarkers associated with the target virus, including epitopes of the target virus, and molecules (e.g. macromolecules, or small molecules) associated with the target virus, including for example, proteins, glycoproteins (e.g., envelope glycoproteins), peptides, polypeptides, polysaccharides, DNA, RNA, and/or antibodies associated with the target virus. Likewise, methods of making the molecularly imprinted polymers of the present disclosure and uses thereof contemplate embodiments wherein the molecularly imprinted polymer selectively binds a portion of a target virus, and/or biomarkers associated with the target virus, including epitopes of the target virus, and molecules (e.g. macromolecules, or small molecules) associated with the target virus, including for example, proteins, glycoproteins (e.g., envelope glycoproteins), peptides, polypeptides, polysaccharides, DNA, RNA, and/or antibodies associated with the target virus.

### DETAILED DESCRIPTION

Viral pandemics are large-scale outbreaks of viral infections that can greatly increase morbidity and mortality over a wide geographic area and cause significant economic, social, and political disruption. Examples of recent viral outbreaks include coronavirus (COVID 2019, MERS 2012, and SARS 2003), influenza viruses (Bird flu H7N9 2013, and Swine flu H1N1 2009), and filoviruses (Zika 2015 and Ebola 2013). The SARS-CoV-2, a betacoronavirus, forms a clade within the subgenus sarbecovirus of the Orthocoronavirinae subfamily. The severe acute respiratory syndrome coronavirus (SARS-CoV) and Middle East respiratory syndrome coronavirus (MERS-CoV) are also betacoronaviruses that are zoonotic in origin and have been linked to potential fatal illness during the outbreaks in 2003 and 2012, respectively.

As of mid-March 2020, the 2019 novel coronavirus (SARS-CoV-2) originating from Wuhan (Hubei Province, China) has infected over 400,000 laboratory-confirmed cases across 188 countries with about 18,000 deaths. Most of the reported cases have similar symptoms at the onset of illness such as fever, cough, and myalgia or fatigue, which progresses in severe cases to pneumonia. In many patients, severe pneumonia results in disease progression to acute respiratory distress syndrome. Based on current evidence, pathogenicity for SARS-CoV-2 is about 4%, which is significantly lower than SARS-CoV (10%) and MERS-CoV (40%). However, SARS-CoV-2 has potentially higher transmissibility (RO: 1.4-5.5) than both SARS-CoV (RO: 2-5) and MERS-CoV (RO: <1). With the possible expansion of SARS-CoV-2 globally and the declaration of the SARS-CoV-2 outbreak as a Public Health Emergency of International Concern by the World Health Organization, there is an urgent need for rapid diagnostics, vaccines and therapeutics to detect, prevent and contain emerging viral outbreaks such as SARS-CoV-2 promptly.

Many viral infection diagnostic tests rely on the detection of antibodies produced as a result of the viral infection. This is an example of indirect recognition since the diagnostic test isn't detecting the virus directly but rather the body's response (the antibody) to the viral infection. Direct recognition would be the detection of the virus or a viral component such as its DNA or RNA sequence or a specific protein. Direct recognition allows for earlier detection since the virus is being detected directly. In the case of indirect recognition and antibody detection, it can take up to several days to weeks (e.g., 1 to 2 weeks) after infection for the body to ramp up antibody levels to the point of detectable limits. However, direct recognition has reduced efficacy late in the infection when recovery is underway. Under these conditions the body has mounted a sufficient immune response reducing the concentration of virus particles in the body. Late in the infection indirect recognition may be preferred because the elevated levels of antibodies will remain in the system well after the infection has been eliminated.

A rapidly developing field for quickly testing patients for viral infection is Rapid Diagnostic Tests (RDTs). RDTs are generally developed from immunoassay techniques and have been used in pregnancy tests, drug tests, HIV detection, influenza detection, strep throat diagnosis, and many others. RDTs are extremely useful as a screening tool to rapidly process large numbers of patients. Often designed to be administered by unskilled technicians, these tests are fast, accurate, and inexpensive relative to other techniques. As a first line of screening these tests can be over 90% accurate and quickly determines if the patient warrants the expenditure of more resources. If positive, the result is validated by a second technique and the necessary medical resources are deployed. For example, RDT diagnostic tests using direct recognition mechanisms for the one or more target viruses of a target viral genus can be deployed to prioritize diagnosing new infections and help contain the spread of the viral agent (for example, using materials, methods and articles disclosed herein to detect the target virus). After release of an initial RDT for the virus, development can continue to add detection capabilities for the antibody to the diagnostic test for example, using materials, methods and articles disclosed herein to detect target antibodies, and/or using a more traditional immunoassay protocol to detect the presence of IgM and IgG antibodies identified for the novel virus. This allows medical professionals to have two to three independent markers for determining infected patients and give bracketed ranges of where they may be in the infection processes (early—no antibodies detected, or late—no virus detected).

The present disclosure relates to a branch of nanotechnology called molecularly imprinted polymers (“MIPs”). The selective binding of MIPs is well known in the literature and have even been referred to as “plastic antibodies”. The MIPs of the present disclosure can be engineered into many forms and can be suspended in an assay, adhered to a wipe, or fixed to a membrane for lateral flow tests. These materials can be specially designed to form a flexible platform that can continuously be altered to meet the demand of detecting constantly evolving viral strains. Having a flexible platform for diagnostics that can easily be adjusted for small structural changes and genetic drift is essential to give healthcare workers tools in real time to effectively mitigate outbreaks.

One of the major advantages of the present technology is that it can utilize off-the-shelf, known chemistries and synthetic ligands for the labeling and visualization of the MIP-bound virus that don't require animal hosts for production. This further accelerates the development timelines. Furthermore, the use of known chemistries and synthetic molecules enhances stability and reduces failed tests due to degradation of biomolecules like antibodies. The use of synthetic ligands also allows for simple modifications in cases where the virus mutates. With antibody-based systems, the labeled antibody will lose its utility if the virus mutates and the binding affinity is lost.

### Target Viruses

The present disclosure provides materials and methods for separating, extracting, or sequestering one or more target analytes (e.g., target virus). In the context of the present disclosure, the target is generally the intact target virus(es). However, the present disclosure also contemplates embodiments where the target analyte is a portion of a target virus, and/or biomarkers associated with the target virus, including epitopes of the target virus, and molecules (e.g. macromolecules, or small molecules) associated with the target virus. Exemplary macromolecules include proteins, glycoproteins (e.g., envelope glycoproteins), peptides, polypeptides, polysaccharides, DNA, RNA, and/or antibodies associated with the target virus. In some embodiments, MIPs of the present disclosure are capable of binding all or a portion of a macromolecule unique to the target virus. For example, the MIPs of the present disclosure are capable of selectively binding to intact SARS-CoV-2. In some embodiments, the MIPs of the present disclosure are capable of selectively binding to an attenuated SARS-CoV-2. In some embodiments, the MIPs of the present disclosure are capable of selectively binding a portion of SARS-CoV-2 (e.g., SARS-CoV-2 spike glycoprotein).

In some embodiments, the MIPs of the present disclosure are prepared by forming an imprint of the virus in a first MIP polymer (using methods as described herein). After removing the virus (or fragments or surrogates thereof) the resulting cavity in the first MIP polymer is used, analogously to a mould, to form a complementary raised shape of substantially the same shape size, and functionality of the virus in a second polymer film. This second polymer film can act as a “stamp” to re-imprint the original viral (or fragments or surrogates thereof) “imprint” from the first MIP polymer, thereby providing a means to rapidly mass-produce MIP materials without the need to use significant quantities of the virus (or fragments or surrogates thereof), and to avoid degradation and other concerns associates with use of the virus.

In some embodiments, the target virus is a novel or emerging target virus. As used herein, the term “novel virus” or “emerging virus” indicates that the virus has a different genetic sequence than any prior virus known in the art. In some embodiments, the target virus is a novel influenza virus or a novel coronavirus. In some embodiments, the target virus is a novel coronavirus. In some embodiments, a novel target virus refers to a virus that has not previously been seen. In some embodiments, a novel target virus can be a virus that is isolated from its natural reservoir or isolated as the result of spread to an animal or human host where the virus had not been identified before. It can be an emergent virus, one that represents a new virus strain, but it can also be an existing virus not previously identified, or a mutant virus of an existing strain.

In some embodiments, the sequence of the target virus or target viral genus comprises about 99%, about 98%, about 95%, about 90%, about 80%, about 70%, about 60%, about 50%, about 40%, about 30%, about 20%, or about 10% polypeptide sequence identity to a known virus, including all values there between. In some embodiments, the sequence of the target virus comprises less than about 99%, less than about 98%, less than about 95%, less than about 90%, less than about 80%, less than about 70%, or less than about 60% polynucleotide sequence identity to a known target virus, including all ranges there between.

In some embodiments, the target virus or target viral genus is a norovirus, rotavirus, adenovirus, astrovirus, influenza virus, coronavirus, parainfluenza virus, respiratory syncytial virus, human immunodeficiency virus (HIV), human T lymphotrophic virus (HTLV), rhinovirus, hepatitis A virus, hepatitis B virus, Epstein Barr virus, or West Nile virus.

In some embodiments, the virus is zika virus, ebola virus, noroviruses, adenoviruses, respiratory syncytial virus (RSV), or human parainfluenza viruses (HPIV) respiratory virus.

In some embodiments, the virus is an influenza virus, including but not limited to any of the types or subtypes, lineages, or clades disclosed herein. There are four types of influenza viruses: A, B, C and D. Human influenza A and B viruses cause seasonal epidemics of disease (known as the flu season) almost every winter in the United States. Influenza A viruses are the only influenza viruses known to cause flu pandemics, i.e., global epidemics of flu disease. Influenza type C infections generally cause mild illness and are not thought to cause human flu epidemics. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). Current subtypes of influenza A viruses that routinely circulate in people include: A(H1N1) and A(H3N2). Certain circulating influenza A(H1N1) viruses are related to the pandemic 2009 H1N1 virus that emerged in the spring of 2009 and caused a flu pandemic. This virus, scientifically called the “A(H1N1)pdm09 virus,” and more generally called “2009 H1N1,” has continued to circulate seasonally since then. Influenza A(H3N2) viruses have formed many separate, genetically different clades in recent years that continue to co-circulate.

In some embodiments, the influenza B virus is classified as further classified as B/Yamagata or B/Victoria.

In certain embodiments, the virus is a coronavirus, including but not limited to any of the types or subtypes or groupings disclosed herein. Coronaviruses are named for the crown-like spikes on their surface. There are four main sub-groupings of coronaviruses, known as alpha, beta, gamma, and delta. Seven coronaviruses that can infect people are: the common human coronaviruses: 229E (alpha coronavirus); NL63 (alpha coronavirus); OC43 (beta coronavirus); HKU1 (beta coronavirus); and other human coronaviruses: MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19). People commonly are infected with human coronaviruses HCoV-229E, NL63, OC43, and HKU1.

COVID-19 is caused by infection with the novel coronavirus, SARS-CoV-2. The SARS-CoV-2 is a spherical enveloped virus with densely glycosylated spike (S) protein used to gain entry into host cells. The spike protein is integral to the infection pathway. The spike protein binds to the cell receptor, angiotensin-converting enzyme 2 (ACE2), by means of the receptor-binding domain (RBD) and undergoes a conformational change to enter the cell. The conformational change of the spike protein is indicative of closely related betacoronaviruses SARS-CoV and MERS-CoV, suggesting that this mechanism is conserved among the Coronviridae. The overall structure of SARS-CoV-2 S resembles that of SARS-CoV S, with a root mean square deviation of 3.8 Å over 959 Ca atoms. Similarly SARS-CoV-2 S shares 98% sequence identity with the S protein from the bat coronavirus RaTG13. Even with it's structural similarities to SARS-CoV S, the SARS-CoV-2 S has a 10-20 fold higher binding affinity to ACE2 at ˜15 nM. The overall structural similarity between the SARS-CoV and SARS-CoV-2 spike protein has led to testing of SARS-CoV RBD-directed monoclonal antibodies (mAbs) for cross-reactivity to the SARS-CoV-2 RBD. Cross-reactivity of the SARS-CoV RBD-directed mAbs has shown almost no affinity to SARS-CoV-2 RBD despite the relatively high degree of structural homology.

In some embodiments, the presently disclosed subject matter relates to one or more molecularly imprinted polymers capable of specifically binding to one or more selected polypeptides and/or portions thereof, wherein each selected polypeptide has an amino acid sequence unique to a specific protein that is uniquely expressed by a specific target virus/or uniquely associated with one or more specific traits of the target virus.

### Molecularly Imprinted Polymers

Host-guest chemistry is used to form materials, such as molecular recognizing polymers (MRP), molecularly imprinted polymers (MIP), and molecular ion sieves, with specifically designed cavities to substantially improve specificity for a “target” analyte (e.g. a target virus) that would be desirable to detect and/or identify in a sample (e.g., biological sample) or to sequester (e.g., isolate) from a sample.

Absorption-based processes are often designed to separate, extract, or sequester a specific molecular species or “target” analyte from a mixture, either to isolate the target analyte (e.g., because of its value), remove a specific species from a mixture (e.g., because of its toxicity or other hazardous properties), or to detect the target analyte (or molecules associated with the target analyte).

Molecularly imprinted polymers are highly selective absorbents with absorption sites specifically tailored to bind to a particular target molecule. Examples of known MIPs and methods of preparing and using MIPs include those disclosed in U.S. Pat. Nos. 7,067,702; 7,319,038; 7,476,316; 7,678,870; 8,058,208; 8,591,842, 9,504,988, 6,582,971, 8,138,289, 6,127,154, 6,582,971, 6,884,842, and 7,285,219 and those disclosed in US publication Nos. 2010/0291224, 2016/0199752, 2010/0297610, 2017/0253647, 2009/0325147, and 2008/0033073 which are incorporated by reference herein in their entirety for all purposes.

In one aspect, the present disclosure is directed to methods for preparing molecularly imprinted polymer (“MIP”) absorbents or materials, MIP absorbents or materials prepared by such processes, and processes utilizing the MIP absorbents or materials of the present disclosure. In some embodiments, the MIP absorbents and materials of the present disclosure are suitable for separating, extracting, or sequestering one or more viruses of a target viral genus, from a sample containing the target virus.

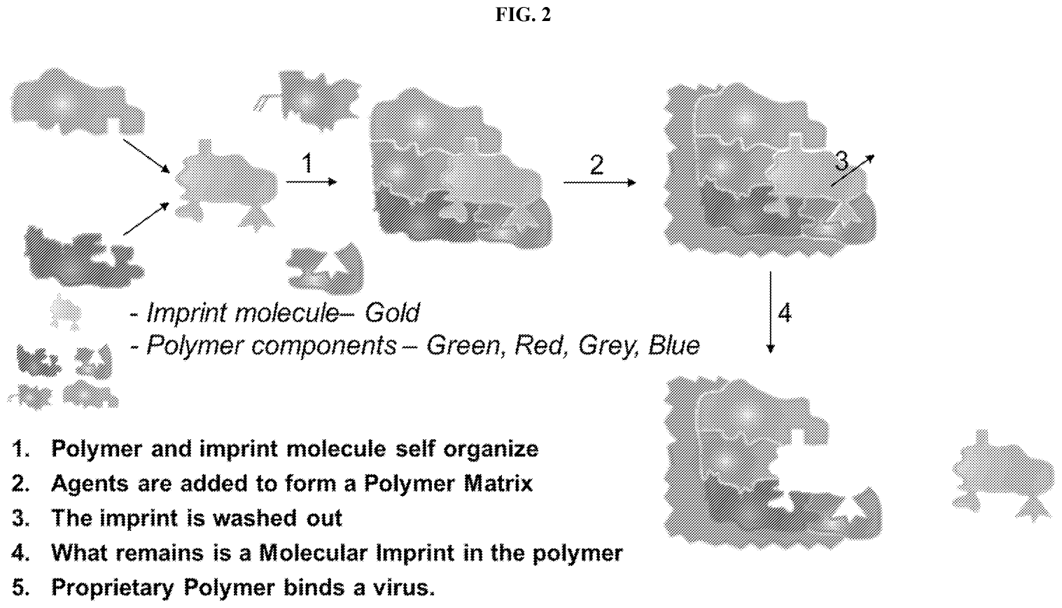

As shown in FIG. 2, MIPs of the present disclosure are prepared by polymerizing a polymerizable ligand(s) which coordinates or non-covalently “binds” with the target virus template (e.g., a “binding monomer”). The target virus and the polymerizable binding monomer are incorporated into a pre-polymerization mixture, allowed to interact and pre-organize, then polymerized (typically in the presence of one or more non-crosslinking monomers and a cross-linking monomer) to form a porous substrate with 3-dimensional cavities and chemistries complementary to the complexed imprint. In some embodiments the non-crosslinking monomer can be omitted, and the MIPs comprise one or more binding monomers and one or more crosslinking monomers. The target virus thus acts as a “template” to define a cavity or binding site within the polymerized matrix which is specific to the target virus (e.g., has a functionality, shape, size and/or morphology corresponding to the target virus). The target virus itself may be utilized to form the MIP or a “surrogate” may be used which mimics the known target (e.g., attenuated target virus). Post-polymerization, the template is then removed from the MIP prior to its use as a selective absorbent for the target virus of interest.

Applicants have found that the selectivity advantages of conventional MIPs can be retained without the need to use the target virus itself as a template for the binding site, by substituting an appropriately selected “surrogate” or “template” for the target virus. As will be exemplified herein, a MIP selective for target virus “A” can be prepared by polymerizing a complex of a suitable surrogate “B” with binding monomer(s) (e.g., charged monomers, uncharged polar monomers and uncharged hydrophobic monomers with functionality complementary to the target virus), in the optional presence of non-crosslinking monomer(s) and the presence of crosslinking monomer(s), provided that “A” and “B” complex to the binding monomer using the same physicochemical mechanism, have similar charge, morphology, size and/or shape, and “B” is one or more of less expensive, less hazardous (i.e., attenuated or inactivated viruses (non-infectious)) or more compatible with the polymerization conditions compared to “A.” In general, the binding monomer will have physicochemical properties suitable for interacting (e.g., through non-covalent interactions) with the virus or biomolecules characteristic of the virus (or fragments thereof or antibodies thereof), such as functional groups that can act as hydrogen bond donors or receptors, functional groups that can participate in hydrophilic or hydrophobic interactions (e.g., Van der Waals interactions), pi-stacking, polar interactions, ionic interactions, non-polar interactions, and the like. In some embodiments, the binding monomer can include multiple functional groups capable of interacting with the virus (or fragments or antibodies thereof) using two or more such interactions. Alternatively (or in combination), the MIP can be prepared from a mixture of different binding monomers, each capable of interacting with the virus (or fragments or antibodies thereof) via different physicochemical interactions. As described herein, virus (or fragment or antibody thereof) surrogates mimic at least some of the characteristics of the target molecule or virus, so the binding monomers would interact with the surrogate in a manner similar to that of the target virus or virus fragment. The resulting “surrogate” templated MIPs, while perhaps somewhat less selective for the target virus than those prepared using the conventional process (in which the target virus serves as the molecular template) are much less expensive, safer to prepare, easier to manufacture and scale-up, etc., yet sufficiently selective in e.g., separation or extraction applications to be similar in performance to conventional MIPs, yet substantially lower in cost. Moreover, the “surrogate” templated MIPs of the present disclosure provide substantial improvements in overall separation process costs due to their combination of high performance at relatively low cost.

Suitable binding monomers have functionality complimentary to the virus of a target viral genus thus, providing an active binding pocket in the final MIP. Various different physicochemical interactions between the binding monomer and the virus of a target viral genus can be exploited to prepare MIPs materials according to the disclosure include covalent, ionic, ion-dipole, hydrogen bonding, dipole-dipole, induced dipole or instantaneous dipole-induced dipole (i.e., London dispersion) attractive interactions, and minimizing coulombic and steric repulsive interactions. Prior to polymerization with one or more uncharged monomers and one or more cross-linking monomers to form the MIP (e.g., MIP bead, or MIP thin film), the binding monomer is mixed with the surrogate virus (or in some embodiments, target virus), which allows the ligand monomer to “self-assemble” or coordinate to the surrogate virus (or target virus) such that during polymerization the surrogate virus (or target virus) is incorporated into the polymerized MIP (e.g. MIP bead, or MIP thin film). As needed, the surrogate virus (or target virus) can be removed from the bead before use by displacement with an appropriate alternative molecule or can remain in place prior to use.

In some embodiments, suitable surrogates can be selected by first characterizing the functionality, morphology, size, shape, and/or relevant physicochemical characteristics of the intact target virus or a portion of the target virus. Candidate surrogates of similar, functionality (e.g., functional group positioning), morphology, size, shape and/or similar physicochemical characteristics can then be identified by various techniques known in the art (e.g. molecular modeling using commercially available molecular modeling programs). Advantageously, the surrogate should be relatively inexpensive, non-toxic, non-virulent, and not interfere with the polymerization (i.e., should not form a highly unstable complex with the binding monomer, poison the polymerization catalyst, inhibit the initiator, react with other monomers or polymerization solvents, be insoluble in the polymerization solvent, etc.). The balancing of these various factors renders the selection of surrogates suitable for various target viruses and separation processes, unpredictable. As will be exemplified herein, suitable surrogates include an attenuated strain of the target virus, or a portion thereof. In some embodiments, the attenuated or inactivated virus retains the shell shape of the virus. In some embodiments, suitable surrogates include all or part of a macromolecule associated with the target virus, such as a polysaccharide group of a glycoprotein macromolecule, or analog thereof. In some embodiments, suitable surrogates include micelles with expressed viral proteins, such as assembled proteins of viral capsid. In some embodiments, the surrogate is an antibody or portion of an antibody of the target virus. In some embodiments, the surrogate is a surface modified dendrimer.

In some embodiments, the MIPs of the present disclosure are in the form of a raised molecularly imprinted polymer stamp having substantially the same shape size, and functionality of the target virus. Such MIPs, are prepared by forming a molecular imprint with the target virus, using techniques known in the art (and described herein), taking the imprint (depression) and imprinting the imprint (depression) into another polymer film giving a “raised stamp” that has the substantially the same shape size and chemistry as the target virus (not complimentary). This “raised stamp” can then be used repeatedly to imprint other MIPs in production without having to worry about degradation of the target virus and other concerns associates with use of the target virus.

Substantially the same morphology, size and shape means that space filling models of the target and the surrogate if superimposed on each other such that the overlap between the volumes defined by the space filling models is maximized (e.g. determined by means of commercial molecular modeling programs) would differ by no more than about 50%, for example, no more than about 50%, no more than about 45%, no more than about 40%, no more than about 35%, no more than about 30%, no more than about 25%, no more than about 20%, no more than about 15%, the more than about 10%, or no more than about 5%, inclusive of all ranges and subranges therebetween.

Alternatively, a surrogate (e.g., attenuated target virus) that is substantially the same size and shape as the target (e.g., target virus) can be functionally defined by the selectivity of the resulting MIP material for the target. Since the complexing cavity of the inventive MIP materials is templated by a surrogate material rather than the target, the selectivity for the MIP material for the surrogate material would be higher than for the target. However, to the extent that the size and shape of the surrogate material would be substantially the same as the size and shape of the target material, the resulting MIP material would have a relatively high selectivity coefficient for the target. Accordingly, higher selectivity coefficients for the target would be indicative that the sizes and shapes of the target and surrogate material are substantially similar. In some embodiments the selectivity coefficient of the MIP materials of the present disclosure for the target, templated with a surrogate of the target, are greater than about 10. In other embodiments, the selectivity coefficient of the MIP materials of the present disclosure are greater than: about 15, about 20, about 25, about 30, about 35, about 40, about 45, about 50, about 100, about 150, about 200, about 300, about 400, about 500, about 600, about 700, about 800, about 900, or about 1000, inclusive of all ranges therebetween.

In some embodiments, the present disclosure provides a molecularly imprinted polymer (MIP) comprising a crosslinked copolymer having a plurality of complexing cavities which selectively binds one or more viruses of a target viral genus, wherein the copolymer is prepared by polymerizing a mixture comprising:

(a) one or more binding monomers complexed to a template having a size, shape and morphology substantially similar to the viruses of the target viral genus;

(b) optionally one or more non-crosslinking monomers; and

(c) one or more crosslinking monomers;

then removing the template from the copolymer, thereby providing said plurality of complexing cavities.

In some embodiments of MIPs of the present disclosure, the target virus is a novel or emerging virus.

In some embodiments, the target viral genus is selected from the group consisting of: norovirus, rotavirus, adenovirus, astrovirus, influenza virus, coronavirus, parainfluenza virus, respiratory syncytial virus, and rhinovirus.

In some embodiments, target viral genus is Betacoronavirus.

In some embodiments of MIPs of the present disclosure, the complexing cavities are selective for SARS-CoV-2.

In some embodiments of the MIPS of the present disclosure, the template is an inactivated form of the target virus. In some embodiments, the template is an inactivated virus of the genus Betacoronavirus. In some embodiments, the template is an inactivated SARS-CoV-2 virus.

In some embodiments of MIPs of the present disclosure, the selectivity of the MIP is at least 70%, including about 70%, about 75%, about 80%, about 85%, about 90%, about 95%, about 96%, about 97%, about 98%, or about 99%, or more. Selectivity, expressed as a percentage, refers to the percentage of complexing cavities occupied by the target (virus or portion of a virus).

In some embodiments of MIPS of the present disclosure, the specificity of the MIP is at least 70%, including about 70%, about 75%, about 80%, about 85%, about 90%, about 95%, about 96%, about 97%, about 98%, or about 99%, or more.

In some embodiments, the present disclosure provides a MIP array comprising two or more MIPs, wherein each MIP comprises a crosslinked copolymer having a plurality of complexing cavities which selectively binds a target virus, wherein the target virus of each MIP is different.

In some embodiments of the MIP arrays of the present disclosure, each crosslinked copolymer is prepared by polymerizing a mixture comprising:

(a) one or more binding monomers complexed to the target virus for that MIP;

(b) optionally one or more non-crosslinking monomers; and

(c) one or more crosslinking monomers;

then removing the virus from the copolymer, thereby providing said plurality of complexing cavities for that MIP.

In some embodiments of the MIP arrays of the present disclosure, the chemical composition of each MIP is the same.

In some embodiments of the MIP arrays of the present disclosure, the chemical composition of at least one MIP is different from the chemical composition of one other MIP.

In some embodiments of the MIP arrays of the present disclosure, the MIP array comprises two or more MIPs of the present disclosure. In some embodiments, the chemical composition of each MIP is the same. In some embodiments, the chemical composition of at least one MIP is different from the chemical composition of one other MIP.

In some embodiments of the MIP arrays of the present disclosure, at least one MIP of the MIP array binds a virus of the genus Betacoronavirus. In some embodiments, at least one MIP of the MIP array binds SARS-CoV-2.

In some embodiments the MIP or MIP arrays of the present disclosure, the sensitivity and selectivity of the at least one MIP that binds a virus of the genus is at least 70% sensitivity and selectivity.

The term sensitivity as used herein, is the proportion of true positives tests out of all patients samples containing the target virus or portion of the target virus. In other words, the extent to which actual positives are not overlooked (so false negatives are few). The equation for sensitivity is the following:

Sensitivity=(True Positives(A))/(True Positives(A)+False Negatives(C)).

As defined herein, Specificity is the percentage of true negatives out of all subjects who do not have a disease or condition. In other words, the extent to which actual negatives are classified as such (so false positives are few). The formula to determine specificity is the following:

Specificity=(True Negatives(D))/(True Negatives(D)+False Positives(B))

For example, if the MIP of the present invention is contacted with 100 samples containing the target virus or portion of the target virus, the detection of the target in 70 of the samples would provide a sensitivity of 70%. Analogously, the percent specificity would be 70% if a MIP of the present invention is contacted with with 100 samples not containing the target virus or portion of the target virus, and correctly provides a negative result (i.e., no detection) of the target in 70 of the samples.

PPVs determine, out of all of the positive findings, how many are true positives; NPVs determine, out of all of the negative findings, how many are true negatives. As the value increases toward 100, it approaches a ‘gold standard’. The formulas for PPV and NPV are shown below.

Positive Predictive Value=(True Positives(A))/(True Positives(A)+False Positives (B))

Negative Predictive Value=(True Negatives(D))/(True Negatives(D)+False Negatives(C))

In some embodiments, the sensitivity of the at least one MIP that binds a virus of the genus is at least about 70%, at least about 75%, at least about 85%, at least about 90%, at least about 95%, at least about 99%. In some embodiments, the sensitivity of the at least one MIP that binds a virus of the genus is about 70%, about 75%, about 85%, about 90%, about 95%, about 99%. In some embodiments, the sensitivity of the at least one MIP that binds a virus of the genus is about 70-99%, about 75-99%, about 80-99%, about 85-99%, about 90-99%, about 95-99%, including all ranges therebetween.

In some embodiments, the selectivity of the at least one MIP that binds a virus of the genus is at least about 70%, at least about 75%, at least about 85%, at least about 90%, at least about 95%, at least about 99%. In some embodiments, the selectivity of the at least one MIP that binds a virus of the genus is about 70%, about 75%, about 85%, about 90%, about 95%, about 99%. In some embodiments, the selectivity of the at least one MIP that binds a virus of the genus is about 70-99%, about 75-99%, about 80-99%, about 85-99%, about 90-99%, about 95-99%, including all ranges therebetween.

In some embodiments of the MIP arrays of the present disclosure, the sensitivity and selectivity of the at least one MIP that binds a virus of the genus Betacoronavirus is at least 70%. In some embodiments, the sensitivity and selectivity of the at least one MIP that binds SARS-CoV-2 is at least 90%.

In various embodiments, the molecularly imprinted polymers are in the form of beads, particularly porous beads that have sufficient porousity so as to allow facile mass transport in and out of the bead.

In some embodiments, the molecularly imprinted polymers of the present disclosure are in the form of a thin film. In some embodiments, the molecularly imprinted polymers of the present disclosure is in the form of a coating.

In some embodiments, the molecularly imprinted polymers of the present disclosure are in the form of molecularly imprinted nano-particles (e.g. imprinted silica nanoparticles).

The complexing cavity may have a geometry (including its size and shape) that is complementary to the template. The shape of the complexing cavity will vary depending on the template. For example, viruses are known to have various shapes, including spherical, icosahedral, or rod-like. As such, where the template is a virus, the shape of the complexing cavity can be spherical, icosahedral, or rod-like. The size of the complexing cavity will also vary depending upon the template. In some cases, the size of the complexing cavity (as measured along its longest axis) can range from 1 nm-2 μm. In some cases, the size of the complexing cavity (as measured along its longest axis) can range from 10 nm-500 nm, complexing cavities having a size in this range may be useful for selectively binding viruses, which generally also have a size in this range. The complexing cavity may be contained in the polymer matrix at any of various locations, including within the polymer matrix (e.g., completely enclosed within the polymer matrix) or at a surface of the polymer matrix (e.g., partially enclosed by the polymer matrix).

For example, MIP beads, according to the present disclosure can have any suitable shape, ranging from approximately spherical, to elongated, irregular (e.g., similar to the irregular shape of cottage cheese curds), or formed to specific desired shapes.

The term “bead” refers to a plurality of particles with an average particle size ranging from about 5 nm to about 1.5 mm. In some embodiments, the average particle size can be about 10-400 nm), nano-MIPs. In some embodiments, the average particle size can be about 250 μm to about 1.5 mm. In some embodiments, the average particle size of the beads can be about 5 nm, about 10 nm, about 15 nm, about 20 nm, about 30 nm, about 40 nm, about 50 nm, about 60 nm, about 70 nm, about 80 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 10 μm, about 15 μm, about 20 μm, about 30 μm, about 40 μm, about 50 μm, about 60 μm, about 70 μm, about 80 μm, about 90 μm, about 100 μm, about 150 μm, about 200 μm, 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, or about 1500 μm, including any ranges between any of these values. In particular embodiments, the average particle size range is from about 0.3 mm to 1.1 mm. In some embodiments, the present disclosure provides a plurality of MIP nanoparticles with an average particle size ranging from about 10 nm to about 400 nm. In some embodiments, the average particle size can be about 5 nm, about 10 nm, about 15 nm, about 20 nm, about 30 nm, about 40 nm, about 50 nm, about 60 nm, about 70 nm, about 80 nm, about 100 nm, about 150 nm, about 200 nm, about 250 nm, about 300 nm, about 350 nm, or about 400 nm. The average particle size of the beads may be measured by various analytical methods generally known in the art including, for example, ASTM D 1921-06, and contact-mode AFM.

In some embodiments, the MIP beads of the present disclosure have a substantially unimodal particle size distribution. In other embodiments, the MIP beads have a bimodal or other multimodal particle size distribution.

The macroreticular polymer beads have a surface area of about 0.1 to about 500 m²/g, for example about 0.1, about 0.5, about 1, about 5, about 10, about 15, about 20, about 30, about 40, about 50, about 60, about 70, about 80, about 90, about 100, about 150, about 200, about 250, about 300, about 350, about 400, about 450, or about 500 m²/g, inclusive of all ranges and subranges therebetween. In some embodiments, the surface area is measured by the Brunauer-Emmett-Teller (BET) method.

In many processes, material handling or mass flow requirements dictate that the percentage of fine particles be low. Accordingly, in particular embodiments, less that about 10% of the MIP beads of the present disclosure have a particle size less than about 250 μm. In other embodiments, less than about 5% or less than about 1% of the beads have a particle size less than about 250 μm.

In some embodiments, the MIP materials (e.g. beads) of the present disclosure are porous to facilitate mass flow in and out of the bead. In some embodiments, the MIP beads of the present disclosure are characterized as “macroreticular” or “macroporous,” which refers to the presence of a network of pores having average pore diameters of greater than 100 nm. In various embodiments, polymer beads with average pore diameters ranging from 100 nm to 2.4 μm are prepared. In some embodiments the average pore diameters can be about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, or about 2400 nm, including ranges between any of these values. Any suitable method for measuring porosity known in the art can be used, for example, in some embodiments, the pore diameter is measured by the Brunauer-Emmett-Teller (BET) method.

The MIP materials can also be mesoporous, or include mesopores (in addition to macropores). The term “mesoporous” refers to porous networks having an average pore diameter from 10 nm to 100 nm. In some embodiments mesopore average pore diameters can be about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, or about 100 nm, including any ranges between any of these values.

Alternatively, the molecularly imprinted polymer can be in a non-bead form, such as nanowires, thin films or membranes, or powders. In particular embodiments, the molecularly imprinted polymer is in the form of a thin film.

In some embodiments, the molecularly imprinted polymer films (recognition layer) has an average layer thickness (e.g., as measured by measured by Atomic Force Microscopy (AFM)) of about 1 nm to about 20 nm. In some embodiments, the average layer thickness is about 2 nm, or about 3 nm, about 4 nm, about 5 nm, about 6 nm, about 7 nm, about 8 nm, about 10 nm, about 11 nm, about 12 nm, about 13 nm, about 14 nm, about 15 nm, about 16 nm, about 17 nm, about 18 nm, or about 20 nm, about 30 nm, about 40 nm, about 50 nm, about 60 nm, about 70 nm, about 80 nm, or about 90 nm. In some embodiments, the thickness of the thin film is measured by Atomic Force Microscopy (AFM).

In some embodiments the MIPs of the present disclosure are in the form of thin films and have a thickness between 1 nm to 100 μm. In some embodiments, the average film thickness is about 1 nm, about 2 nm, or about 3 nm, about 4 nm, about 5 nm, about 6 nm, about 7 nm, about 8 nm, about 10 nm, about 11 nm, about 12 nm, about 13 nm, about 14 nm, about 15 nm, about 16 nm, about 17 nm, about 18 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 420 nm, about 430 nm, about 440 nm, about 450 nm, about 460 nm, about 470 nm, about 480 nm, about 490 nm, about 500 nm, about 600 nm, about 700 nm, about 800 nm, about 900 nm, about 1 μm, about 10 μm, about 20 μm, about 30 μm, about 40 μm, about 50 μm, about 60 μm, about 70 μm, 8 about 80 μm, about 90 μm, to about 100 μm, including all values and subranges therebetween. In some embodiments, the MIPs of the present disclosure are in the form of thin films have a thickness between about 20 nm to about 1 μm. In some embodiments the MIPs of the present disclosure are in the form of thin films have a thickness between about 1 to about 500 nm. In some embodiments, MIPs of the present disclosure (e.g., MIP QCM sensor) are in the form of thin films have a thickness between about 100 nm to about 300 nm. In some embodiments, the thickness of the thin film is measured by Atomic Force Microscopy (AFM).

In some embodiments, molecularly imprinted polymers (e.g., MIP thin film) are prepared, wherein the imprinted layer (i.e., the approximate depth of the imprint or complexing cavity) has a depth ranging from about 10 nm to 200 nm, including about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, including all values and subranges therebetween. In some embodiments, MIP materials of the present disclosure (e.g., MIP thin films) have an imprint cavity (or complexing cavity) ranging from about 5 nm to about 100 μm in diameter, including from about 5 nm, about 10 nm, about 15 nm, about 20 nm, about 30 nm, about 40 nm, about 50 nm, about 60 nm, about 70 nm, about 80 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 1.5 μm, about 2 μm, about 3 μm, about 5 μm, about 10 μm, about 15 μm, about 20 μm, about 30 μm, about 40 μm, about 50 μm, about 60 μm, about 70 μm, about 80 μm, about 90 μm, to about 100 μm in diameter, including all subranges and values therebetween. In some embodiments, the MIP materials have an imprint cavity ranging from about 5 nm to about 500 nm in diameter, or from about 50 nm to about 100 nm in diameter. Any suitable method for measuring the diameter of the imprint cavity known in the art can be used, for example Atomic Force Microscopy (AFM).

In addition, the MIP materials can also be microporous, or include micropores in addition to macropores and/or mesopores. The term “microporous” refers to porous networks having an average pore diameter less than 10 nm. In some embodiments micropore average pore diameters can be about 0.5 nm, about 1 nm, about 1.5 nm, about 2 nm, about 2.5 nm, about 3 nm, about 3.5 nm, about 4 nm, about 4.5 nm, or about 5 nm, or about 5.5 nm, about 6 nm, about 6.5 nm, about 7 nm, about 7.5 nm, about 8 nm, about 8.5 nm, about 9 nm, about 9.5 nm, or about 10 nm, including ranges between any of these values. Any suitable method for measuring porosity known in the art can be used, for example Atomic Force Microscopy (AFM).

The structure and porosity of the beads are determined principally by the conditions of polymerization. The desired porosity of the bead can be achieved by the choice of surrogate/binding monomer complex, optional non-crosslinking monomer and crosslinking monomers and their amounts, as well as the composition of the reaction solvent(s) and optional pore forming additives or thixotropic agents. Porosity determines the size of the species, molecule or ion that may enter a specific structure and its rate of diffusion and exchange, as well as the rate of mass flow in and out of the bead structure.

The thixotropic agents can significantly improve control of bead formation and substantially uniform bead or particle size. Suitable thixotropic agents employed herein are dependent on the type and amount of monomer employed and the suspending medium. The thixotropic agents can also advantageously act as suspension agents during the suspension polymerization process. Representative examples of such thixotropic agents include, but are not limited to, cellulose ethers such hydroxyethyl cellulose, (commercially available under the trade name of “CELLOSIZE”), cross-linked polyacrylic acid such as those known under the name of “CARBOPOL” polyvinyl alcohols such as those known under the trade name of “RHODOVIOL”, boric acid, gums such as xanthan gum and the like and mixtures thereof, The amount of thixotropic agents can influence the size of the resin (i.e., the use of larger amounts of thixotropic agents often results in the formation of smaller resin particles).

The amount of the thixotropic agent is generally from about 1.5 to about 5 weight percent, based on the weight of the monomers in the monomer mixture. In some embodiments, the amount of the thixotropic agent is from about 1.5 to about 2.5 weight percent, based on the weight of the monomer or monomers (combination of monomers) in the monomer mixture.

In some embodiments, the resistance across the circuit on the QCM crystal in the presence of MIPs of the present disclosure (e.g., MIP QCM sensor) under PBS buffer (static conditions) is measured before the addition of virus. In some embodiments, the resistance across the circuit on the QCM crystal is less than about 2000 Ohms, less than about 1500 Ohms, less than 1000 Ohms, or less than about 500 Ohms. In some embodiments, the baseline resistance under PBS buffer of the MIPs is less than about 1000 Ohms. In some embodiments, the resistance across the circuit on the QCM crystal ranges from about 300 Ohms to about 2000 Ohms, or about 300 Ohms to about 1000 Ohms, including all values and ranges therebetween.

Cross-linking (also crosslinking) agents or cross-linking monomers that impart rigidity or structural integrity to the MIP are known to those skilled in the art, and include di-, tri- and tetrafunctional acrylates or methacrylates, divinylbenzene (DVB), alkylene glycol and polyalkylene glycol diacrylates and methacrylates, including ethylene glycol dimethacrylate (EGDMA) and ethylene glycol diacrylate, vinyl or allyl acrylates or methacrylates, diallyldiglycol dicarbonate, diallyl maleate, diallyl fumarate, diallyl itaconate, vinyl esters such as divinyl oxalate, divinyl malonate, diallyl succinate, triallyl isocyanurate, the dimethacrylates or diacrylates of bis-phenol A or ethoxylated bis-phenol A, methylene or polymethylene bisacrylamide or Bismuth-acrylamide, including hexamethylene bisacrylamide lanthanide or hexamethylene bismethacrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1, 1-dimethy 1-2-isocyanaotoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, divinyl benzene; 1,3-divinyltetramethyl disiloxane; 8,13-divinyl-3,7,12,17-tetramethyl-21H,23H-porphine; 8,13-divinyl-3,7,12,17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; divinyl tin dichloride; N,N′-methylenebisacrylamide, N,N′-bisacryloyl-1,2-dihydroxy-1,2-ethylenediamine (DHEBA); Ethylene dimethacrylate; tetraethyl orthosilicate; piperazine diacrylamide; aminophenylboronic acid (APBA); 3-Aminopropyltriethoxysilane; (3-Aminopropyl)-trimethoxysilane (APTMS); Ethylene glycoldimethacrylate (EGDMA; trimethylolpropane trimethacrylate (TRIM); polydimethylsiloxane (PDMS), polyacrylate, silica (SiO₂), and polyurethane (PU) and phloroglucinol; and the like.

In some embodiments, the cross-linking agent or cross-linking monomer is selected from the group consisting of divinylbenzene (DVB); N,N′-methylenebisacrylamide; N,N′-bisacryloyl-1,2-dihydroxy-1,2-ethylenediamine (DHEBA); Ethylene dimethacrylate (EDMA); tetraethyl orthosilicate (TEOS); piperazine diacrylamide; aminophenylboronic acid (APBA); 3-Aminopropyltriethoxysilane (APTES); polydopamine (PDA); (3-Aminopropyl)-trimethoxysilane (APTMS); Ethylene glycoldimethacrylate (EGDMA; trimethylolpropane trimethacrylate (TRIM); polydimethylsiloxane (PDMS); polyacrylate, silica (SiO₂); polyurethane (PU); pentaerythritol triacrylate and phloroglucinol.

In some embodiments, crosslinking monomers used for preparing a MIP of the present disclosure are one or more difunction acrylate, methacrylate, acrylamide, or other suitable monomers, for example monomers selected from the group consisting of ethylene glycol dimethacrylate (EGDMA), N,N′-methylenebisacrylamide, N,N′-(1,2-dihydroxyethylene)bisacrylamide, and combinations thereof.

In some embodiments, a MIP of the present disclosure is prepared by polymerizing a mixture comprising from about 1% (w/w) to about 90% (w/w) crosslinking monomer(s) relative to total monomer, including about 1% (w/w), about 5% (w/w), 10% (w/w), about 15% (w/w), about 20% (w/w), about 25% (w/w), about 30% (w/w), about 35% (w/w), 40% (w/w), about 45% (w/w), about 50% (w/w), about 55% (w/w), about 60% (w/w), about 65% (w/w), to about 70% (w/w), about 75% (w/w), about 80% (w/w), about 85% (w/w), to about 90% (w/w) including all values and subranges therebetween relative to total monomer, from about 40% (w/w) to about 70% (w/w) crosslinking monomer(s) relative to total monomer.

The MIP must have sufficient rigidity so that the target analyte or surrogate analyte may be easily removed without affecting the integrity of the polymer. In such cases where the polymer matrix is insufficiently rigid, crosslinking or other hardening agents can be introduced. In some embodiments, in imprinted MIPs described herein, the cross-linker (cross-linking agent or monomer) fulfills three major functions: 1) the cross-linker is important in controlling the morphology of the polymer matrix, whether it is gel-type, macroporous or a microgel powder; 2) it serves to stabilize the imprinted binding site (complexing cavity); and 3) it imparts mechanical stability to the polymer matrix. In particular embodiments, high cross-link ratios are generally desired in order to provide permanently porous materials with adequate mechanical stability.

A wide variety of monomers may be used as binding monomers, or optional non-crosslinking monomers for synthesizing the MIP in accordance with the present disclosure. Suitable non-limiting examples of binding monomers or non-crosslinking monomers that can be used for preparing a MIP of the present disclosure include methylmethacrylate, other alkyl methacrylates, alkylacrylates, allyl or aryl acrylates and methacrylates, cyanoacrylate, styrene, substituted styrenes, methyl styrene (multisubstituted) including 1-methylstyrene; 3-methylstyrene; 4-methylstyrene, etc.; vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy) ethyl methacrylate; 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; aeryloyl chloride; 1-α-acryloyloxy-β,β-dimethyl-γ-butyrolactone; N-acryloxy succinimide acryloxytris(hydroxymethyl)amino-methane; N-acryloyl chloride; N-acryloyl pyrrolidinone; N-acryloyl-tris(hydroxymethyl)amino methane; 2-aminoethyl methacrylate; N-(3-aminopropyl)methacrylamide; (o, m, or p)-amino-styrene; t-amyl methacrylate; 2-(1-aziridinyl)ethyl methacrylate; 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; -bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; t-butylacrylamide; butyl acrylate; butyl methacrylate; (o, m, p)-bromo styrene; t-butyl acrylate; 1-carvone; (S)-carvone; (−)-carvyl acetate; 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloroethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 1-(3-butenyl)-4-vinylbenzene; 2,4-dimethyl-2,6-heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstyrene; 3,4-dimethylstryene; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethy 1-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); GA GMA; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; (±)-linalool; linalyl acetate; p-mentha-1,8-diene; p-mentha-6,8-dien-2-ol; methyleneamino acetonitrile; methacrolein; [3-(methacryloylamino)-propyl] trimethylammonium chloride; methacrylamide; methacrylic acid; methacrylic anhydride; methacrylonitrile; methacryloyl chloride; 2-(methacryloyloxy)ethyl acetoacetate; (3-meth-acryloxypropyl)trimethoxy silane; 2-(methacryloxy)ethyl trimethylammonium methylsulfate; 2-methoxy propene (isopropenyl methyl ether); methyl-2-(bromomethyl)acrylate; 5-methyl-5-hexen-2-one; methyl methacrylate; N,N′methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; methyl vinyl ether; methyl-2-vinyloxirane; methyl vinyl sulfone; 4-methyl-5-vinylthiazole; myrcene; t-nitrostyrene; 3-nitrostyrene; 1-nonadecene; 1,8-nonadiene; 1-octadecene; 1, 7-octadiene; 7-33ctane-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyanoethylene; trans 3-chloroacrylic acid; 2-trifluoromethyl propene; 2-(trifluoromethyl)propenoic acid; 2,4,4′-trimethyl-1-pentene; 3, 5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethylsulfone; 4-(vinyl benzyl)-2-chloroethyl sulfone; N-(p-vinylbenzyl)-N,N′-dimethyl amine; 4-vinyl biphenyl (4-phenylstyrene); vinyl bromide; 2-vinyl butane; vinyl butyl ether; 9-vinyl carbazole; vinyl carbinol; vinyl cetyl ether; vinyl chloroacetate; vinyl hloroformate; vinyl crotanoate; vinyl peroxcyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinylquinoline; vinyl iodide; vinyllaurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbomene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinylquinoline; 1-vinyl silatrane; vinyl sulfone; vinyl sulfonic acid sodium salt; a-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy) silane; vinyl 2-valerate; vinyl benzoic acid; and vinyl imidazole, dopamine (DA); and the like.

In some embodiments, suitable binding monomers for synthesizing MIPs in accordance with the present disclosure include acrylamide, methacrylic acid, methylmethacrylate, N-vinylpyrrolidone, acrylic acid, N-benzylacrylamide, Ethylene glycol dimethacrylate (EDMA); tetraethyl orthosilicate (TEOS); piperazine diacrylamide; 3-aminophenylboronic acid (APBA); 3-Aminopropyltriethoxysilane (APTES); polydopamine (PDA); N-methacryloyl-L-tyrosine methyl ester; hydroxyethylmethacrylate (HEMA); N-Isopropylacrylamide (NIPAM); acrylic acid (AA); polyurethane (PU); bisphenol A; phloroglucinol; N-tert butyl acrylamide; poly(tert-butylmethacrylate)-block-poly(2-hydroxyethyl methacrylate); allylamine; 1-vinyl imidazole; aminoethylacrylamide; (3-acrylamidopropyl) trimethyl ammonium chloride; N-(3-aminopropyl)methacrylamide; N-tert-butyl acrylamide; N-phenylacrylamide; N-hydroxymethylacrylamide; epichlorohydrin; sulphonic acids (e.g., 2-acrylamido-2-methylpropane sulphonic acid); carboxylic acids (e.g., acrylic acid, vinylbenzoic acid); heteroaromatic bases (e.g., vinylpyridine, vinylimidazole); polyvinylpyrrolidone (PVP), dimethylamino; ethyl methacrylate (DMAEMA), and polyamine (PA)

In some embodiments, the monomer is N-vinylpyrrolidone, methylmethacrylate, methacrylic acid, acrylamide and the cross-linking agent is N,N-(1,2-dihydroxyethylene) bisacrylamide.

In some embodiments, binding monomers used for preparing a MIP of the present disclosure include one or more binding monomers selected from the group consisting of acrylic acid, methacrylic acid, acrylamide, methacrylamide, vinylpyridine, N-alkylacrylamides, N-isopropylacrylamide, hydroxyethylmethacrylate, and any combination thereof.

In some embodiments, non-crosslinking monomers used for preparing a MIP of the present disclosure include one or more non-crosslinking monomers are selected from the group consisting of alkylmethacrylates, methylmethacrylate, ethylmethacrylate, and combinations thereof.

In some embodiments, a MIP of the present disclosure is prepared by polymerizing a mixture comprising about 1% (w/w) to about 90% (w/w) binding monomer(s) or non-crosslinking monomer(s) relative to total monomers, including about 1% (w/w), about 5% (w/w), 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), to about 70% (w/w), about 75% (w/w), about 80% (w/w), about 85% (w/w), to about 90% (w/w) including all subranges and values therebetween. In some embodiments, the mixture comprises about 30% (w/w) to about 50% (w/w) binding monomer(s) or non-crosslinking monomer(s) relative to total monomer.

Acrylate-terminated or otherwise unsaturated urethanes, carbonates, and epoxies can also be used in the MIP. An example of an unsaturated carbonate is allyl diglycol carbonate. Unsaturated epoxies include, but are not limited to, glycidyl acrylate, glycidyl methacrylate, allyl glycidyl ether, and 1,2-epoxy-3-allyl propane.

In some embodiments, a MIP of the present disclosure is prepared by polymerizing a mixture further comprising viscosity modifier. In some embodiments, the viscosity modifier is a polymer selected from the group consisting of poly(acrylic acid), poly(ethylene oxide), poly(ethyleneimine), poly(propylene oxide), copolymers of ethylene oxide and propylene oxide, block copolymers of polyethylene oxide (PEO) and polypropylene oxide (PPO), triblock PEO-PPO-PEO polymers, and combinations thereof. In some embodiments, the viscosity modifier is poly(acrylic acid). In some embodiments, the polymer viscosity modifiers have a molecular weight (Mw) ranging from about 10,000 g/mol to about 1,000,000 g/mol, including from about 10,000 g/mol, about 50,000 g/mol, about 100,000 g/mol, about 150,000 g/mol, about 200,000 g/mol, about 250,000 g/mol, about 300,000 g/mol, about 350,000 g/mol, about 400,000 g/mol, about 450,000 g/mol, about 500,000 g/mol, about 550,000 g/mol, about 600,000 g/mol, about 650,000 g/mol, about 700,000 g/mol, about 750,000 g/mol, about 800,000 g/mol, about 850,000 g/mol, about 900,000, to about 1,000,000, including all values and subranges therebetween. In some embodiments, the polymer viscosity modifiers have a molecular weight (Mw) ranging from about 50,000 to about 500,000 g/mol.

In some embodiments, a MIP of the present disclosure is prepared by polymerizing a mixture comprising between about 0.1% (w/w) and about 30% (w/w) viscosity modifier relative to total monomer, including between about 0.1% (w/w), about 0.5% (w/w), about 1% (w/w), about 1.5% (w/w), about 2% (w/w), about 2.5% (w/w), about 3% (w/w), about 4% (w/w), about 5% (w/w), about 6% (w/w), about 7% (w/w), about 8% (w/w), about 9% (w/w), about 10% (w/w), about 15% (w/w), about 20% (w/w), about 25% (w/w), and about 30% (w/w), including all values and subranges therebetween.

In some embodiments, a MIP of the present disclosure is prepared by polymerizing a mixture comprising about 0.1% (w/w) to about 5% (w/w) photoinitiator relative to total monomer, including about 0.1% (w/w), about 0.5% (w/w), about 1.0% (w/w), to about 1.5% (w/w). In some embodiments, the mixture comprises about 0.5 (w/w) % to about 1.5% (w/w) photoinitiator.

In some embodiments, a MIP of the present disclosure is prepared by polymerizing a mixture comprising about 40% (w/w) to about 70% (w/w) crosslinking monomer, about 30% (w/w) to about 50% (w/w) binding monomer and crosslinking monomer, and about 0.5% (w/w) to about 1.5% (w/w) photoinitiator relative to total monomer.

In some embodiments, a MIP of the present disclosure is prepared by polymerizing a mixture comprising about 40% (w/w) to about 70% (w/w) crosslinking monomer, about 30% (w/w) to about 50% (w/w) binding monomer and crosslinking monomer, and a catalytic photoinitiator relative to total monomer.

In some embodiments, a MIP of the present disclosure is prepared by polymerizing a mixture comprising about 40% (w/w) to about 70% (w/w) crosslinking monomer, about 30% (w/w) to about 50% (w/w) binding monomer and crosslinking monomer, about 0.5% (w/w) to about 5% (w/w) viscosity modifier, and a catalytic photoinitiator relative to total monomer.

The MIPs of the invention can be prepared in accordance with any technique known to those skilled in the art using a target virus, target virus surrogate or a portion thereof as a template molecule.

The beads of the present disclosure can be prepared by various polymerization techniques. A polymer matrix can then be formed via a suitable polymerization technique in the presence of the surrogate/binding monomer complex to form an imprinted resin. The resin product can be then be recovered. Non-limiting examples of suitable polymerization techniques can include aqueous polymerization, precipitation polymerization; miniemulsion polymerization; aqueous suspension polymerization, inverse suspension polymerization (e.g. in perfluorocarbon), non-aqueous dispersion polymerization, two-stage swelling polymerization, aerosol polymerization, latex seeded emulsion polymerization, electropolymerization, and bulk polymerization on porous bead substrates. In some embodiments, the polymerization method is the aqueous suspension polymerization of a copolymerizable mixture of an organic phase containing non-ligand monomer, an optional crosslinker, and the surrogate/binding monomer complex, and an aqueous phase containing at least one or more thixotropic agents. In some embodiments, the polymerization method is aqueous polymerization and the aqueous polymerization can be carried out without denaturing the template.

In some embodiments, the MIPs of the present disclosure are in the form of nanoMIPs, which may be prepared by various polymerization techniques including but not limitated to precipitation polymerization (PP), (mini- or micro)emulsion polymerization (EP), solid-phase imprinting (SPI), and surface imprinting (SI) (on nanoparticles). In addition, some other special approaches such as nanoprecipitation imprinting (NpI), self-assembly imprinting (SAI), and monomolecular imprinting (MmI) (inside dendrimers) are also available for preparing nanoMIPs.

In some embodiments, provided herein is a method of preparing a molecularly imprinted polymer (MIP) comprising a crosslinked copolymer having a plurality of complexing cavities which selectively binds one or more viruses of a target viral genus, wherein the copolymer is prepared by polymerizing a mixture comprising:

(a) one or more binding monomers complexed to a template having a size, shape and morphology substantially similar to the viruses of the target viral genus;

(b) optionally one or more non-crosslinking monomers; and

(c) one or more crosslinking monomers;

then removing the template from the copolymer, thereby providing said plurality of complexing cavities.

In some embodiments, provided herein is a method of preparing a molecularly imprinted polymer (MIP) thin film comprising a crosslinked copolymer having a plurality of complexing cavities which selectively binds one or more viruses of a target viral genus wherein the MIP thin film is prepared by

(a) polymerizing a mixture comprising:
i) one or more binding monomers;
ii) optionally one or more non-crosslinking monomers, and
iii) one or more crosslinking monomers;

thereby forming an intermediate copolymer;

(b) coating the intermediate copolymer on a solid support to form a thin film,

(c) pressing a template having a size, shape and morphology substantially similar to the viruses of the target viral genus; into the surface of the thin film; and

(d) curing the thin film complexed to the template to form a crosslinked copolymer;

then removing the template from the copolymer, thereby providing said plurality of complexing cavities.

In some embodiments, the virus template is frozen prior to templating. In some embodiments, the virus template is bound to a glass slide using a fixing agent (e.g., glutaraldehyde).

In some embodiments, the polymerization mixture comprises an organic solvent. Suitable organic solvents include, one or more solvents selected from the group consisting of ethanol, isopropanol, THF, acetonitrile, and DMSO.

Thin films of MIPs of the present disclosure may be obtained, for example, using controlled radical polymerization (CRP) to produce a thin film polymer at an interface where one of the phases (liquid, solid or gas) can be removed after polymerization and be replaced with another phase (liquid, solid or gas). In some embodiments the polymerization is carried out by grafting under controlled radical polymerization conditions (CRP) of one or several monomers by the “grafting to” technique or by the “grafting from” technique. The CRP may be performed by atom transfer radical polymerization (ATRP), relying on redox reactions between alkyl halides and transition metal complexes; by stable free radical polymerization (SFRP) making use of initiators decomposing to one initiating radical and one stable free radical or by radical addition fragmentation chain transfer (RAFT) polymerization.

In some embodiments, a MIP (e.g., MIP film) is prepared by providing an aqueous pre-polymerization mixture containing, for example, one or more crosslinking monomers, one or more binding monomers, optionally one or more non-crosslinking monomers, and optionally adjusting the pre-polymerization mixture to a pH of about 7, adding an initiator and pre-polymerizing the mixture (e.g. under UV light) until the gel point is reached, optionally diluting the solution, coating (e.g., spin coating, doctor-blading, transfer coating, etc.) the solution on a surface to form a thin film, and stamping the surface with a template (e.g., target virus, portion of the target virus, biomarker of target virus or surrogate thereof) followed by polymerization.

In some embodiments of the present disclosure, a MIP (e.g., MIP nanoparticle) is prepared by providing an aqueous mixture containing one or more crosslinking monomers, one or more binding monomers, optionally one or more non-crosslinking monomers, and optionally adjusting the pre-polymerization mixture to a pH of about 7. An initiator is then added to the pre-polymerization mixture with a template (e.g., target virus, portion of the target virus, biomarker of target virus or surrogate thereof). The resulting mixture can be pre-polymerized under UV light, for example, until the gel point was reached, then diluted (e.g., in any suitable solvent, such as water and acetonitrile) and rapidly stirred. In some embodiments, the crosslinking agent is N,N′-(1,2-dihydroxyethylene) bisacrylamide, the binding monomers are methacrylic acid, and vinylpyrrolidone, and the radical initiator is potassium peroxydisulphate. In certain embodiments of the present disclosure, a MIP is prepared by miniemulsion polymerization. In the miniemulsion polymerization procedure, one or more binding monomers, optionally one or more non-crosslinking monomers, and one or more crosslinking monomers are dispersed in a continuous phase with the application of high shear forces. A co-surfactant may be added to suppress diffusion processes occurring in the continuous phase in order to create a stable emulsion with homogeneous sizes. The template molecule (e.g., target virus, portion of target virus, biomarker of target virus or surrogate thereof) is added to into the miniemulsion and mixed with a magnetic stirrer to allow interaction between template and binding monomers. The resulting mixture is diluted and polymerized (e.g., at 40° C.). Polymerization times can vary (e.g., from about 3 hours to about 72 hours), depending on the reactivity of the monomers. Miniemulsion polymerization may produce about 1:1 transfer of monomer nanodroplets into regularly shaped polymerized nanobeads (e.g., at least about 50 nm in size).

In some embodiments of the present disclosure MIPs in the form of a film coating a core support shell is prepared by miniemulsion polymerization. In the miniemulsion polymerization procedure support beads (e.g., (poly(MMA-co-EGDMA)) may be synthesized in a first stage core-shell miniemulsion polymerization followed by optional functionalization (e.g., by aminolysis and aldehyde functionalization of aminolysis, aldehyde functionalization of poly(MMA-co-EGDMA support beads). The template (e.g., target virus, portion of the target virus, biomarker of target virus or surrogate thereof) is immobilized on the support particles, and a second stage miniemulsion polymerization is initiated to form a polymeric shell over the template-immobilized core particles. The template may be removed using any suitable conditions known in the art, including for example, base hydrolysis to provide a templated MIP shell having complementary target cavities specific to the template molecule.

In some embodiments of the present disclosure MIPs are prepared by aqueous polymerization. For example, a binding monomer/surrogate complex of the present disclosure can be polymerized under aqueous polymerization conditions where the aqueous solution comprises, for example, the polymerizable binding monomer/surrogate complex, binding monomer (e.g., APTES, TEOS, APTMS etc.), cross-linking agent (e.g., glutaraldehyde), aqueous solvent (e.g. PBS, water etc), and an initiator (e.g. APS). The aqueous solution is agitated, for example shaken with vertical rotations. By varying the temperature, agitation, polymerizable ligand/surrogate loading, solvent ratios, and degree of cross-linking, different structures and properties can be obtained.

In certain embodiments of the present disclosure, a MIP is prepared by suspension polymerization of a surrogate/binding monomer complex and other monomers as described herein. In the suspension polymerization procedure, the various phases can be thoroughly mixed separately prior to the start of the reaction and then added to the polymerization reaction vessel. While this mixing of the ingredients can be done in a vessel other than the reaction vessel, the mixing can alternatively be conducted in the polymerization reaction vessel under an inert atmosphere, particularly where the monomers being employed are subjected to oxidation. Further, in order to improve yields and selectivity of the final resin product, it is desirable that the ligand monomer be hydrolytically stable under polymerization conditions and in the final product. For example, the ligand monomer can be hydrolytically stable in a suspension polymerization formulation and under a water treatment environment such that hydrolysis is substantially avoided during polymerization and the useful life of the resin.

The polymerizable binding monomer/surrogate complex of the present disclosure can be polymerized under suspension polymerization conditions where the aqueous phase contains thixotropic agents such as polyvinyl alcohol and boric acid in water, and the organic phase comprises, for example, the polymerizable binding monomer/surrogate complex, styrene (non-crosslinking monomer), divinylbenzene (cross-linking monomer), organic solvents, and AIBN (initiator). The biphasic mixture is agitated, for example with a stirrer. By varying the temperature, agitation, polymerizable ligand/surrogate loading, solvent ratios, and degree of cross-linking, different beads structures and properties can be obtained. For example, spherical and porous beads of the desired size can be obtained by controlling the agitation or stirring during the polymerization. When the polymerization mixture is agitated to disperse the monomers dissolved in the organic reaction medium as droplets within the aqueous phase, suitably the droplets are of such size that when transformed into polymer beads, they are substantially spherical and porous, and of the desired size. Unsuitable reaction conditions can lead to the formation of no or very small beads, high surrogate losses to the aqueous phase, low overall yield, and insufficient porosity such that there is poor mass transfer to the complexing cavity.

Proper and sufficient agitation or stirring throughout the polymerization typically provides substantially spherical and porous beads having the desired size. For example, the polymerization mixture can be agitated to disperse the monomers (dissolved in the solvent organic phase) in the aqueous solvent phase by shear action, thereby forming droplets. By selecting the proper level of agitation, the droplets can be of such size that when transformed into polymer beads, they are substantially spherical and porous, and will have the desired size as discussed herein.

Various means are available to maintain the proper agitation. When polymerization is conducted in a reactor made of stainless steel, such a reactor can be fitted with a rotatable shaft having one or more agitator blades. When a round-bottom flask is used as a reactor, an overhead stirrer can be used to agitate the reaction medium. The amount of agitation necessary to obtain the desired results will vary depending upon the particular monomers being polymerized, as well as the particular polymer bead size desired. Therefore, the agitation speed such as the rpm (revolutions per minute) may be regulated within certain limits. Polymerization times can vary (e.g., from about 3 hours to about 72 hours), depending on the reactivity of the monomers.

When polymerization is complete, the surrogate can be removed from the typically cross-linked polymer beads without substantially affecting the complexing cavity. Removal of the surrogate molecule provides e.g. a bead having a porous structure with complementary molecular cavities therein that has high binding affinity for the target virus, portion of the target virus, and/or unique biomarker of the target virus.

Any suitable conditions effective to polymerize the monomers of the present disclosure to produce an MIP without dissociating the binding ligand/surrogate complex may be used.

The monomers of the present disclosure may be polymerized by free radical polymerization, and the like. Any photoinitiators (e.g., photoinitiator for UV polymerization) or thermal free radical initiator known to those skilled in the art can be used in the preferred free radical polymerization. Examples of suitable photoinitiators (e.g., photoinitiator for UV polymerization) and thermal initiators include benzoyl peroxide, acetyl peroxide, lauryl peroxide, azobisisobutyronitrile (AIBN), t-butyl peracetate, cumyl peroxide, t-butyl peroxide; t-butyl hydroperoxide, bis(isopropyl) peroxy-dicarbonate, benzoin methyl ether, 2,2′-azobis(2,4-dimethyl-valeronitrile), tertiary butyl peroctoate, phthalic peroxide, diethoxyacetophenone, t-butyl peroxypivalate, diethoxyacetophenone, 1-hydroxycyclohexyl phenyl ketone, 2,2-dimethyoxy-2-phenylacetophenone, and phenothiazine, diisopropylxanthogen disulfide, 2,2′-azobis-(2-amidinopropane); 2,2′-azobisisobutyronitrile-; 4,4′-azobis-(4-cyanovaleric acid); 1,1′-azobis-(cyclohexanecarbonitrile)-; 2,2′-azobis-(2,4-dimethyl valeronitrile); and the like and mixtures thereof. In some embodiments, peroxodisulfates (ammonium peroxodisulfates (APS) or potassium peroxodisulfates (KPS)) with N,N,N,N-tetramethylethylenediamine (TEMED) are used as an initiator and catalyst that may be used in free radical polymerization to produce MIPs of the present disclosure. In some embodiments, the photoinitiator is selected from the group consisting of azoisobutyronitrile (AIBN), diphenyl(2,4,6-trimethylbenzoyl)phosphine oxide (TPO), 1-phenyl 1,2-propanedione (PPD), camphorquinone (CQ), and phenylbis(2,4,6-trimethylbenzoyl)phosphine oxide, and combinations thereof. In some embodiments, the photoinitiator is a photoinitiator that can be activated at a non-germicidal wavelength. In some embodiments, the UV wavelength is greater than about 300 nm. In some embodiments, the UV wavelength is between about 280 nm and about 400 nm, including about 280 nm, about 285 nm, about 290 nm, about 295 nm, about 300 nm, about 305 nm, about 310 nm, about 315 nm, about 320 nm, about 325 nm, aout 330 nm, about 335 nm, about 340 nm, about 345 nm, about 350 nm, about 355 nm, about 360 nm, about 365 nm, about 370 nm, about 375 nm, about 380 nm, about 385 nm, about 390 mm about 395 nm, and about 400 nm, including all values and subranges therebetween. In some embodiments, the UV wavelength is between about 280 nm and about 315 nm, or between about 315 nm and about 400 nm.

The choice of monomer and cross-linking agent will be dictated by the chemical (hydrophilicity, chemical stability, degree of cross-linking, ability to graft to other surfaces, interactions with other molecules, etc.) and physical (porosity, morphology, mechanical stability, etc.) properties desired for the polymer. The amounts of binding monomer/surrogate complex, monomer and crosslinking agents should be chosen to provide a crosslinked polymer exhibiting the desired structural integrity, porosity and hydrophilicity. The amounts can vary broadly, depending on the specific nature/reactivities of the binding monomer/surrogate complex, monomer and crosslinking monomer chosen as well as the specific application and environment in which the polymer will ultimately be employed. The relative amounts of each reactant can be varied to achieve desired concentrations of binding monomer/surrogate complexes in the polymer support structure. Typically, the amount of binding monomer surrogate complex will be on the order of about 0.01 mmol to about 100 mmol percent of monomer, including: about 0.02, 0.05, 0.1, 0.2, 0.3, 0.5, 1, 2, 3, 4, 5, 10, 15, 20, 25, 30, 35, 40, 45, 50, 55, 60, 65, 70, 75, 80, 85, 90, 95, or 100 mmol percent of monomer. The amount of cross-linker is typically on the order of about 1.0 to about 10 mole percent, including about 1.5, 2, 3, 4, 5, 6, 7, 8, or 9 mole percent of monomer. The amount of a free radical initiator can be about 0.005 to 1 mole percent, including about 0.01, 0.05, 0.1, 0.5, 0.6, 0.7, 0.8, or 0.9 mole percent of monomer. (Molar percentages refer to the percentage relative to the total amount of monomers prior to polymerization.)

As such the binding monomer as described herein comprises all or nearly all of the monomer used in preparing the MIP with little to no supporting polymer backbone and crosslinking. Such a binding monomer must be functionalized and soluble in the conditions of suspension polymerization and must still result in a final polymerized form that maintains the polymer qualities suitable for commercial use (rigidity, selectivity, reuse capability, temperature and pH resistance). MIP materials of the present invention are stable (physically and chemically) in a pH range of about 0-13 (including about 0, 1, 2, 3, 4, 5, 6, 7, 8, 9, 10, 11, 12, and 13, inclusive of all ranges therebetween), a temperature range of about 0-100° C. (including about 0, 10, 20, 30, 40, 50, 60, 70, 80, 90, 100° C., inclusive of all ranges therebetween), have a mass attrition of less than about 20 wt. % (including less than about 20, 15, 10, 9, 8, 7, 6, 5, 4, 3, 2, 1, 0.5, or approximately 0 wt. %, inclusive of all ranges therebetween), stability to at least about 20 you cycles (including about 2, 3, 4, 5, 6, 7, 8, 9, 10, 11, 12, 13, 14, 15, 16, 17, 18, 19, or 20, inclusive of all ranges therebetween) and a selectivity coefficient (as described herein) for the desired target molecule of at least about 40 (including at least about 40, 45, 50, 55, 60, 65, 70, 75, 80, 85, 90, 95, or 100, inclusive of all ranges therebetween). The solvent, temperature, and means of polymerization can be varied in order to obtain polymeric materials of optimal physical or chemical features, for example, porosity, stability, and hydrophilicity. The solvent will also be chosen based on its ability to solubilize all the various components of the reaction mixture and form a desirable polymer morphology.

The degree of crosslinking can range from about 1% to about 95%. In some embodiments, the degree of crosslinking is from about 5% to about 80%.

Any solvent which provides suitable solubility and is compatible with the desired reaction to the conditions to form the MIP materials of the present disclosure may be used. For example, the solvent can include long chain aliphatic alcohols such as pentanols, hexanols, heptanols, octanols, nonanols, decanols, undecanols, dodecanols, including saturated and unsaturated isomers thereof (e.g., methyl and ethyl pentanols, methyl and ethyl hexanols, methyl and ethyl, heptanols, etc.), aliphatic hydrocarbons (e.g., butanes, pentanes, hexanes, heptanes, etc.), aromatic hydrocarbons (e.g., benzene, toluene, xylenes, etc.). Suitable solvents also include DMSO, acetonitrile, phosphate buffer (e.g., phosphate buffer pH 4, phosphate buffer pH 8.0 etc.), water, Tris-HCl buffer pH 8.5, toluene, MOPS buffer pH 6.0, ethanol, Tris-HCl, THF, or combinations thereof. Suitable adsorption solvents, include but are not limited to, PBS (e.g., pH 7.2, pH4.0), water (e.g., distilled or ultrapure), Tris-HCl (e.g., pH 6.2-7.2), and phosphate buffer (e.g., pH 8.0).

The resin thus obtained is in the form of porous beads. Porous beads can have an open cell structure such that the majority of open volumes within the bead are interconnected with one another and external openings on surfaces of the bead.

The present disclosure provides methods for preparation of MIPs. MIPs can be prepared by modification of known techniques including but not limited to those described in U.S. Pat. Nos. 4,406,792, 4,415,655, 4,532,232, 4,935,365, 4,960, 762, 5,015,576, 5,110,883, 5,208,155, 5,310,648, 5,321,102, 30 5,372,719, 5,786,428, 6,063,637, and 6,593,142, and U.S. application Ser. No. 15/176,158; the entire contents of each of which are incorporated herein by reference in their entireties for all purposes.

A variety of MIPs in the form of thin films are suitable for use in the present disclosure. For example, a hydrogel composed of a material such as dextran may serve as a suitable MIP film. A variety of techniques may be used to generate regions of MIP film on the surface of the support or on the surface of a coating on the support. These techniques are well known to those skilled in the art and will vary depending upon the nature of the MIP film, and the support. Areas of MIP film may be created by microfluidics printing, microstamping (U.S. Pat. Nos. 5,731,152 and 5,512,131), or microcontact printing (U.S. Pat. No. 6,180,239), others focus on using polymeric gels as bulk imprinting matrices. In some embodiments of the present disclosure MIPs may be formed by imprinting techniques selected from bulk imprinting (3D), surface imprinting (2D), soft lithography, self-assembly, or by Core-shell particles via immobilized templates as described, for example, in Gast et al. Trends in Analytical Chemistry 114 (2019) 218e232, which is incorporated herein by reference.

In some embodiments, the MIPs of the present disclosure comprise organic, or inorganic polymers or pre-polymerized polymers. Polymers can include polystyrene (PS); polyacrylates; polymethacrylates; polyvinylpyrrolidone (PVP); polyacrylamides; polyurethanes; and Epon1002F; polypyrroles (PPY); poly(ethylene-co-vinyl alcohol); organosiloxane polymers generated by sol-gel chemistry; ethylene glycol dimethacrylate; poly(vinylpyrrolidoneco-methacrylic acid); poly(vinylpyrrolidoneco-methacrylic acid) crosslinked with N,N′-(1,2-dihydroxyethylene)-bisacrylamide (DHEBA); Polyallylamine/ethylene glycol diglycidyl ether (EGDE); poly(hydroxylethyl-methacrylate-N-methacryloyl-L-tyrosine methylester; orPolyallylamine/ethylene glycol diglycidyl ether (EGDE); or combintions thereof.

“Hydrogels” include but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), cross-linked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

In some embodiments, the MIPs of the present disclosure are prepared by Microcontact stamping (or microprinting) using methods known in the art. Microcontact stamping involves deposition of the target (e.g. a target virus, or in other embodiments, a surrogate) onto a flat solid support layer (and then topping them with monomers or a soft polymer such as prepolymerized polyurethane (PU). The polymer is then cured, sandwiching the target (or surrogate) between the support layer and the formed polymer. Microcontact stamping can be performed using both organic and inorganic polymers.

In some embodiments, microprinting involves generating a polymer layer using target virus or surrogate of the target virus as a template, and then using this as a mould to generate a second polymer layer. This layer can then act as a ‘master mould’ that can be used as a template instead of the target virus or surrogate. This may improve the ease, reproducibility, and safety of making imprinted polymer layers because no living virus is needed after the first imprint.

In some embodiments of the disclosure, MIPs are prepared by viral stamping. In exemplary viral stamps comprise self assembled virus layers at different concentrations ranging from 1 to 1000 μgmL⁻¹ on very flat glass substrates such as glass slides (roughness approximately 2 nm). Such viral stamps may be prepared by applying the virus suspension to the glass surface which are then assembled at 4° C.

For example, exemplary MIPs of the present disclosure may be prepared using conditions known in the art, for example disclosed in Med. Chem. Commun., 2014, 5, 617-62). Such MIPs may be obtained, for example, by providing a pre-polymerization mixture of acrylamide (e.g. 13 mg), methacrylic acid, (e.g. 10 mg), methylmethacrylate (e.g. 6 mg), and N-vinylpyrrolidone (6 mg) dissolved in dimethylsulfoxide (e.g. 300 mL) containing the initiator 2,2-azobis(isobutyronitrile). The pre-polymerization mixture is pre-polymerized to reach a gel point (e.g. by heating at 70 C). The pre-polymerization polymers may be dropped on to a surface (e.g., QCM electrode) and spun off to obtain a thin layer. The template stamp is prepared on a glass substrate by adding the virus sample (e.g., 5 ml of virus sample, kept at 4 C for 30 minutes). Then, the template stamp is pressed onto the polymer layer and polymerized under UV light (254 nm) overnight. The imprinted cavities of the virus were obtained by removing the virus template from the rigid polymer with 10% hydrochloric acid and stirred in water.

In some embodiments of the present disclosure, functionalized silica beads (e.g., functionalized with amino- and glutaraldehyde-groups) are used as core particles for imprinting MIPs disclosed herein. In typical imprinting protocols functionalized silica beads are suspended (e.g., 10 mg in a aqueous solution such as PBS e.g. 4 mg/mL) by ultra-sonication. The beads are incubated with a template (e.g., target virus or surrogate thereof), then monomers and crosslinkers were added (such as TEOS, APTES, and APTMS e.g. 10 μL: 2 μL: 2 μL). The polymerization step was performed by agitating the mixture (e.g. for 3 h at 4° C. and 700 rpm). To remove the template from the imprinted particles, the beads were incubated with a clearing solution containing 1M HCl and 0.01% Triton X-100 under rotating conditions (e.g., 60 rpm, RT), centrifuged and washed with PBS.

In some embodiments of the present disclosure, MIPs are coated on glass microbeads. In a typical protocol, glass beads are washed and incubated in a 5% v/v glutaraldehyde solution in PBS (pH 7.2) for e.g., 2 h, and rinsed with double-distilled water. Templates (e.g., target virus or surrogate thereof) are immobilized by incubating the glass beads with a solution of the template in PBS (pH 7.2) overnight at 4° C. The beads are rinsed with water and dried. A 50 mL solution of ethanolamine (0.1 mM) in PBS (pH 7.4) is optionally used to incubate the glass beads for 15 min, and washed with double distilled water before being used.

In some embodiments, a pre-polymerization mixture of monomers disclosed herein (e.g., NIPAm (53% mol), BIS (2% mol), TBAm (40% mol), and AAc (5% mol) are dissolved in an aqueous solution (e.g., water). The total monomer concentration in the polymerization mixture is about 6.5 mM. The solution is degassed under vacuum and sonicated, and then purged with N₂. The pre-polymerization mixture is contacted with the template-derivatized glass beads in the optional presence of an additional monomer (e.g., N-(3-Aminopropyl) methacrylamide to functionalize primary amine groups to the surface of the MIPs nanoparticle). Polymerization is started by adding an initiator to the agitated solution (e.g. aqueous solution (e.g., 800 μL) of stock solution of APS (180 mg) and, 90 μL of TEMED in water (e.g. 3 mL)).

In some embodiments, the MIPs of the present disclosure are prepared by epitope imprinting using methods known in the art which focuses particular molecular components on the cell surface of target viruses such as proteins, lipids, saccharides, and their derivatives. In some embodiments, MIPs of the present disclosure are prepared by imprinting a small peptide sequence, or epitope, that is characteristic of a particular target virus and exposed on its surface. Access of the target to the sites may be obtained by immobilizing the template on a “void creator” such as porous silica gel, or colloidal silica which post-polymerization, is removed. Alternatively, the epitope may be conjugated to a polymeric protein surrogate that is then removed for liberation of the epitopeselective sites.

In some embodiments, the MIPs of the present disclosure comprise organosilane polymers, for example organosilane polymers deposited on a substrate (e.g., silica, glass, or the surface of a device such as a quartz crystal microbalance). Crosslinking monomers can include halosilanes or alkoxysilanes, such as tetrachlorosilane, tetramethoxy- or tetraethoxysilane (TEOS), and binding monomers such as aminopropyltriethoxysilane (APTES), bis[(3-triethoxysilyl)]propylamine, bis[(3-trimethoxysilyl)]propylamine, 3-aminopropylmethyldiethoxysilane, 3-aminopropylmethyldimethoxysilane, aminoethylaminopropyltrimethoxysilane, aminoethylaminopropyltriethoxysilane, aminoethylaminopropylmethyldimethoxysilane, diethylenetriaminopropylmethyldimethoxysilane, piperazinylpropylmethyldimethoxysilane, n-propyltriethoxysilane, benzyltriethoxysilane and combinations thereof. Such monomers can be polymerized under aqueous conditions, in the presence of the target virus or surrogate thereof (as described herein) to provide a silsequioxane polymer layer with a plurality of complexing cavities.

In some embodiments, MIPs of the present disclosure are prepared, by imprinting the virus on the surface of stable, nano-sized thin polymer films (e.g., silsesquioxane films) formed on the surface of silica nanoparticles, using methods and techniques known in the art, and described for example in (B. Yang et al. Biosensors and Bioelectronics 87 (2017) 679-685, Cumbo et al. Nature Communications volume 4, Article number: 1503 (2013); Gast, M. et al. Anal. Chem. 2018, 90, 5576-5585). For example, in some embodiments, MIPs of the present disclosure may be prepared by surface imprinting techniques utilizing silica nanoparticles that act as a carrier material and organosilanes. In some embodiments, the surface of silica nanoparticles (SNPs) modified with a low density of amine groups at the silica nanoparticles surface in a manner that allows control of thickness and composition. Such MIPs may be obtained, generally, by (1) binding the template target virus or target virus surrogate on the surface of a silica nanoparticles carrier material (e.g., under aqueous conditions using a crosslinking agent e.g. glutaraldehyde) (2) incubating the virus-modified nanoparticles with a mixture of organosilanes, followed by the polycondensation of the latter to grow an organosilica (silsesquioxane) recognition layer thereby forming a replica of the virus; and (iii) removing the template to free the imprints. Removal of the template virus from the MIP can be achieved using any suitable condition known in the art, including for example suspension in acidic solution (e.g., 1M HCl and 0.01% v/v Triton-X 100).

Various MIP materials of the present disclosure can be reused (regenerated) more than once and frequently up to about 30 times or more, depending on the particular resin and the treated liquid medium. Regeneration can be accomplished in much the same manner as removal of the original imprint ion, e.g. stripping or washing with an appropriate solution.

In other embodiments, the MIP materials are not regenerated.

Materials comprising a molecularly imprinted polymer of the present disclosure (e.g., macroreticular MIP beads, MIP films, MIP coatings and/or MIP nanoparticles) are particularly useful for selectively removing or adsorbing target viruses, portion of the target virus, unique biomarker of the target virus or surrogates thereof.

The MIP materials (e.g., beads or macroreticular beads) according to the present disclosure are selective for the target. The selectivity of the MIP material to bind species “A” in a mixture of “A” and specie “B” can be characterized by a “selectivity coefficient” using the following relationship:

$$Selectivity coefficient for A = \frac{[ {A}^{′} ] [ B ]}{[ A ] [ {B}^{′} ]}$$

where “[A]” and “[B]” refer to the molar concentration of A and B in solution, and “[A′]” and “[B′]” refer to the concentration of complexed “A” and “B” in the MIP material.

For most separations, the selectivity coefficient for the target virus, portion of the target virus, and/or unique biomarker of the target virus versus other species in the mixture to be separated should be at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 70, at least about 80, at least about 90, at least about 100, at least about 200, at least about 300, at least about 400, at least about 500, including ranges between any of these values.

As used herein, the term “bind,” “binding,” “bond,”, “bonded,” or “bonding” refers to the physical phenomenon of chemical species being held together by attraction of atoms to each other through sharing, as well as exchanging, of electrons or protons. This term includes bond types such as: ionic, coordinate, hydrogen bonds, covalent, polar covalent, or coordinate covalent. Other terms used for bonds such as banana bonds, aromatic bonds, or metallic bonds are also included within the meaning of this term. The selective binding interactions refer to preferential and reversible binding exhibited by the MIP for a target molecule as described herein.

Throughout the description, where methods or processes are described as having, including, or comprising specific process steps, the processes also consist essentially of, or consist of, the recited processing steps. Further, it should be understood that the order of steps or order for performing certain actions is immaterial so long as the method remains operable. Moreover, two or more steps or actions can be conducted simultaneously.

### Uses of Molecularly Imprinted Polymers

In some embodiments, the molecularly imprinted polymer is initially complexed to a template having a size, shape and morphology substantially similar to the viruses of the target viral genus (e.g., inactivated form of the target virus) and before use, the template is stripped out of the molecularly imprinted polymer. In some embodiments, the stripping comprises contacting the molecularly imprinted polymer with an acidic solution (e.g., aqueous inorganic acid or organic acid, such as HCl, acetic acid) to denature the virus and washing with deionized water to remove/elute the virus. In some embodiments, the stripping step comprises a salt water wash.

In some embodiments, the present disclosure provides a microwell comprising a MIP of the present disclosure.

In some embodiments, the present disclosure provides a cartridge comprising a container filled with a MIP of the present disclosure.

In some embodiments, the present disclosure provides a sensor coated with at least one MIP of the present disclosure, wherein when the MIP coating binds to the target virus, such binding causes a change in sensor properties capable of providing a detectable signal.

In some embodiments, the present disclosure provides a quartz crystal microbalance resonator coated with at least one MIP of any of the present disclosure.

In some embodiments, the present disclosure provides an article comprising a MIP of the present disclosure and further comprising a labeling composition comprising a label, wherein the label binds to any target virus bound to the MIP.

In some embodiments, the present disclosure provides an article comprising a MIP of the present disclosure, and a labeling composition comprising a label, wherein the label binds to any target virus bound to the MIP.

In some embodiments, the present disclosure provides an article comprising a MIP (as disclosed herein) comprising a crosslinked copolymer having a plurality of complexing cavities which selectively bind a target virus and a labeling composition comprising a label, wherein the label binds to any target virus bound to the MIP, wherein said MIP is prepared by polymerizing a mixture comprising:

(a) one or more binding monomers complexed to the target virus;

(b) optionally one or more non-crosslinking monomers; and

(c) one or more crosslinking monomers;

then removing the virus from the copolymer, thereby providing said plurality of complexing cavities.

In some embodiments of the MIP array, each crosslinked copolymer is prepared by polymerizing a mixture comprising:

(a) one or more binding monomers complexed to an inactivated form of the target virus for that MIP; and
(b) one or more crosslinking monomers; and
(c) removing the inactivated virus from the copolymer, thereby providing said plurality of complexing cavities for that MIP.

In some embodiments the article of the present disclosure is in the form of a fabric, face mask, swab, coating, badge, or sprayable liquid, which when contacted with the target virus or virus of a target viral genus, binds the labeling compound to any target virus or virus of a target genus bound to the MIP.

In some embodiments of the article of the present disclosure, the labeled target virus or labeled virus of a target genus bound to the MIP is detectable colorimetrically, by fluorescence, or magnetically.

Some embodiments relate to a method of selectively sequestering one or more viruses of a target viral genus present in a sample, comprising contacting a molecularly imprinted polymer of the present disclosure with a sample comprising one or more viruses of a target viral genus, thereby selectively sequestering the one or more viruses of a target viral genus in the molecularly imprinted polymer. In some embodiments, the present disclosure provides methods of detecting a target virus or a target viral genus comprising:
(a) contacting a sample with a MIP of the present disclosure, thereby binding at least a portion of any viruses of the target viral genus present in the sample to the MIP; and
(b) reacting a label molecule capable of attaching to the bound viruses;
wherein the label molecule can be detected to show the presence of the bound virus.

In some embodiments, the present disclosure provides methods of detecting a target virus or a target viral genus comprising:
(a) contacting a sample with an article of the present disclosure, thereby binding any of the target viruses in the sample to one or more of the MIPs; and
(b) reacting a label molecule capable of attaching to the bound viruses;
wherein the label molecule can be detected to show the presence of the bound target virus.

In some embodiments of the methods disclosed herein the label molecule is a fluorophore (e.g., a green emitting fluorophore, or a red emitting fluorophore). In some embodiments, the label molecule is an amine-specific fluorophore. In some embodiments, the label molecule is a succinimidyl ester (such as Alexa Fluor 488 NHS ester). In some embodiments, the fluorophore is an Alexa Fluor dye (e.g., Alexa Fluor 488, or Alexa Fluor 594). In some embodiments, the label molecule is detected by a fluoresence microscope.

In some embodiments, the methods of the present disclosure further comprise stripping the one or more target viruses out of the molecularly imprinted polymer. In some embodiments, the stripping step comprises contacting the molecularly imprinted polymer bound to one or more target viruses with acidic solution (e.g., aqueous inorganic acid or organic acid, such as HCl, acetic acid) to denature the virus and washing with deionized water to remove/elute the virus. In some embodiments, the stripping step comprises a salt water wash.

In some embodiments, the sample is any sample containing or potentially containing a target virus. In some embodiments, the sample is selected from the group consisting of aerosols, fluid samples, solid samples, biological samples, insects, food matrices, and environmental samples containing or potentially containing a target virus. In some embodiments, the sample is droplets or fomites generated by, e.g., exhaled breath, coughing and/or sneezing which contains or potentially contains a target virus.

In some embodiments, the labeled target virus or labeled virus of a target genus bound to the MIP is detectable by any suitable methods known in the art, including, for example, colorimetrically, by fluorescence, or magnetically. In some embodiments, the labeled target virus bound to the MIP is detected colorimetrically. In some embodiments, the labeled target virus bound to the MIP is detected by fluorescence.

In some embodiments, the presence of one or more viruses of a target viral genus in a sample may be detected by independent molecular recognition and detection events. In some embodiments, the presence of one or more viruses of a target viral genus in a sample may be detected by molecular recognition coupled to a specific detection event. In some embodiments, the presence of one or more viruses of a target viral genus in a sample may be detected by Cartridge type molecular recognition with primary or secondary detection.

In some embodiments, the detection limit of the methods and devices disclosed herein is about about 1 ng/mL to about 100 ng/ml, including all values therebetween. In some embodiments, the detection limit of the methods and devices disclosed herein is about about 1 ng/mL. In some embodiments, the detection limit of the methods and devises disclosed herein is about 10 micrograms/ml.

In some embodiments, the detection limit of the MIPs of the present disclosure is at least about 100 target virus particles/mL, at least about 500 target virus particles/mL, at least about 1000 target virus particles/mL, at least about 1500 target virus particles/mL, at least about 2000 target virus particles/mL, at least about target virus 5000 particles/mL, at least about 10,000 target virus particles/mL, at least about 15,000 target virus particles/mL, at least about 1×10⁶ target virus particles/mL, or at least about 1×10⁸ target virus particles/mL. In some embodiments, the detection limit of the MIPs of the present disclosure is at least about 105 target virus particles/mL. In some embodiments, the detection limit of the MIPs of the present disclosure is at least about 15,000 target virus particles/mL. In some embodiments, the detection limit of the MIPs of the present disclosure is at least about 1×10⁶ target virus particles/mL.

In some embodiments, the presence of one or more viruses of a target viral genus in a sample is detected directly, for example, by the binding of the virus to a labeled antibody conjugate. There are two readily available options to prepare a labeled antibody. In one embodiment, the labeled antibody is prepared by adsorbing the antibody on gold nanoparticles (colloidal gold) which provides a distinct red to orange color change depending on the gold particles utilized. This is a well-known process and is common in lateral flow and other immunoassays. A useful feature is it is applicable to any protein, enzyme antibody. In some embodiments, the labeled antibody is prepared by conjugating the antibody to a chromophore or fluorescent entity using standard protein conjugation chemistry known in the art (e.g. water soluble carbodiimide couplings, conjugation of activate ester forms of chromophores/fluorophores). In some embodiments, an amine terminated dendrimer is utilized as the detection group conjugated to the antibody. In this format, the dendrimer can be pre-labeled with multiple dye molecules which may enhance sensitivity.

In some embodiments, the presence of one or more viruses of a target viral genus in a sample is detected, for example, by the binding of the virus to a labeled virus binding protein (determined by medical/research community). The labeling process would be similar to antibodies, with the use of gold nanoparticles (e.g., colloidal gold) or dye conjugates to add a detectable group.

In some embodiments, the presence of one or more viruses of a target viral genus in a sample is detected by the binding of the virus to a labeled synthetic ligand of the target virus (nature of ligand determined by medical/research community).

If no known ligands or other biological entities are known or available during initial RDT development, the presence of the virus may be visualized utilizing known amine/protein labeling techniques. Known chemistries are highly reactive and provide a high level of sensitivity based on the dye choice although they are not specific for the target virus. The highly selective nature of the MIP/virus binding will minimize false positive results using this process.

The presence of the virus may also be determined indirectly by the binding of an anti-virus antibody (IgG and IgM as per the medical community) to a labeled antigen entity (determined by medical/research community).

In some embodiments the present disclosure provides a sensor for detecting a virus, coated with a MIP or MIP array of the present disclosure. In some embodiments, the sensor is selected from a quartz crystal electrode, SPR plate, electrochemical sensor, magnetic particle, or optical fiber.

In some embodiments, the present disclosure provides methods of detecting a target virus with a sensor of the present disclosure, wherein the sensor is exposed to a sample, whereby target virus in the sample binds to the MIP or MIP array coating, and the amount of target virus in the sample correlates to a change in sensor response to said binding of the target virus to the MIP or MIP array.

In some embodiments, the present disclosure provides a method of removing one or more target viruses from a fluid, comprising contacting the fluid with a MIP or MIP array of any of the present disclosure, whereby at least a portion of any target virus in the fluid binds to the MIP or MIP array.

In some embodiments of the methods of removing one or more target viruses, the fluid is water. In some embodiments, the fluid is air.

In some embodiments of the methods of removing one or more target viruses, the MIP or MIP array is in the form of a container filled with the MIP or MIP array.

In some embodiments of the methods of removing one or more target viruses, the bound target virus is quantified.

The present disclosure also provides methods of diagnosing a subject infected with a target virus utilizing MIPs of the present disclosure, in addition to diagnostic kits are provided. In one aspect, the present disclosure provides a kit comprising a one or more MIPs of the present disclosure for detecting or identifying all or part of a target virus, and/or unique biomarkers associated with a target virus. In some embodiments, the present disclosure provides methods for determining the onset, progression, or regression of an infection associated with a target virus a subject, wherein a biological sample obtained from a subject is screened for said virus by contacting said biological sample with one or more MIPs of the present disclosure having an affinity for the target virus. In one aspect, the present disclosure provides a kit comprising one or more MIPs for detecting or identifying target virus particles.

The present invention, also provides methods for detecting the presence of a target virus on a target area, comprising contacting the target area with one or more MIPs having an affinity for the target virus. Exemplary target areas include environmental surfaces such as hospitals, medical devices, floor sweepings, bed railings, tables, bedding, cloths, and door knobs etc.

In embodiments, testing occurs occurs at the onset of infection. In embodiments, testing occurs after the subject is infected with the target virus but prior to the onset of symptoms of the target virus. Accordingly, in some embodiments, testing occurs after the subject is identified as having been infected with the target virus (e.g., SARS-CoV-2) or after the subject has been identified as being suspected of being infected with the target virus, or as being at risk of infection with the target virus. In embodiments, testing occurs prior to infection with the virus. In embodiments, the testing occurs prior to the onset of symptoms of the viral infection. In embodiments, testing occurs after the onset of symptoms of the viral infection (e.g., respiratory viral infection).

### Technology Platform:

The present disclosure, relates in part, to a platform technology that can be rapidly applied to detect emerging viral threats once information is available from the medical community. To rapidly develop a new diagnostic in response to a new outbreak, and as disclosed herein, the present disclosure provides a family of MIPs based on the general, functionality (e.g., functional group positioning) shape, size and morphology of generic classes of viruses. As described herein, these MIPs are prepared utilizing all or part of the target virus or utilizing in various embodiments, surrogates, or attenuated or inactivated strains of the target viruses (non-infectious). The MIPS are part of an inventory ready to be immediately deployed for screening against a new viral threat. For example, in some embodiments, the target viral threat is SARS-CoV-2 and the present disclosure provides a MIP that recognizes/binds all or a portion of SARS-CoV-2.

In general, a target virus identified by the medical community, is screened against the viral MIP library for the most selective binding interaction in collaboration with a Virology Laboratory. Once synthesized and processed the MIPs will be tested in non-competitive and competitive binding experiments to determine capacity, robustness and selectivity. These experiments is/are designed to give a general idea about the performance parameters of the MIPs and give a feedback loop for optimizing the MIP formulation.

In parallel with the synthesis of new MIPs is the development of the labeling and binding chemistry (e.g., as described herein). Once an imprint has been selected and there is a set of ligands showing preliminary affinity towards the imprint, the specific chemistry for the molecular tags can be determined. This chemistry is dependent on the nature of the imprint.

After the MIP has been selected and synthesized the next stage is to process it to apply the MIP formulation to a target device or platform. Development of the binding of the recognition site (i.e. the MIP) to the device substrate will be required and will be dependent on the nature of the polymer matrix synthesized. In some embodiments, the MIP formulation can be applied to multiple devices and give reproducible results.

The final phase is testing of samples containing or suspected of containing one or more target virus(es). Here samples will be tested with the prototype devices and tested for accuracy, sensitivity, selectivity, and speed. Training requirements for technicians will also be determined at this stage, but with a colorimetric design there should be minimal training time required. Successful clinical validation will lead to scale-up and mass production with an appropriate partner that was involved in the prototyping stage as well.

In some embodiments, the diagnostic tests and/or articles disclosed herein, can be labeled with a quick response (QR) code, or other suitable machine-readable labels known in the art. Such labels can be used, for example, in connection with a device (e.g., mobile app or other suitable devices known in the art) that can read the label, and automatically verify the presence of labeled target viruses bound to MIPs of the present disclosure and upload the diagnostic information to a central database. For example, in some embodiments, the labeled target virus bound to a MIP of the present disclosure can be detected colorimetrically and the colorimetric response can be processed as an image by a software algorithm to verify any color change and the resulting data transmitted to a central database.

Throughout the description, where methods or processes are described as having, including, or comprising specific process steps, the processes also consist essentially of, or consist of, the recited processing steps. Further, it should be understood that the order of steps or order for performing certain actions is immaterial so long as the method remains operable. Moreover, two or more steps or actions can be conducted simultaneously.

### EXAMPLES

### Example 1. Virus Imprinted MIPs

MIPs of the present disclosure are formed by producing a formulation comprising monomers with chemical functionalities that will self-assemble and associate with the virus surface. In some embodiments, these formulations are polymerized into oligomers that form a pre-polymer gel. The pre-polymer gel is processed into a thin film (e.g., by spin coating, doctor blading, or comparable techniques) on a solid support substrate such as a glass slide or a quartz crystal microbalance electrode.

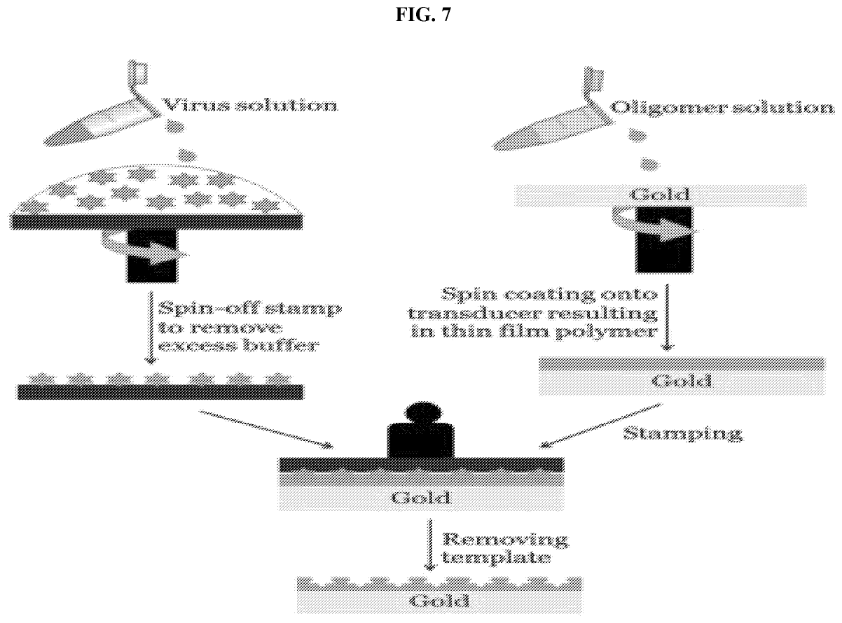

A typical imprinting processes is depicted in FIG. 7, and consists of securing the template (live virus, inactivated virus, or alternative surrogate) to a solid support substrate (such as a glass slide) to create a “stamp”. Deposition of the template onto the solid support substrate needs to be single particle thickness with no aggregates and is primarily controlled by virus concentration in solution (e.g., volumes of 5 μL-10 μL and concentrations of 1-1000 μg/mL for making the imprint stamp). The imprint stamp is pressed into the pre-polymer gel and polymerized with a secondary reaction to yield a polymeric thin film. After the reaction is complete the imprint stamp is removed and washed to clean the surface and remove any templates that detached during the process.

Characterization of the surface imprinting and the formed cavities/mold can be done using atomic force microscopy (AFM), scanning electron microscopy (SEM), and transmission electron microscopy (TEM). These techniques will verify the size, shape, and placement density of the cavities on the MIP surface.

The MIP is tested against simple solutions (buffer and virus) of the virus (either live or inactive) with the benchmark limit of detection being 15,000 particles/mL. The binding efficiency is determined using a quartz crystal microbalance (QCM) and/or a multistep processes to yield a colorimetric response. The MIP processed onto the QCM electrode is inserted into a flow cell configuration where solutions containing the virus can be passed over the MIP and the binding measured. Buffer solution, virus solution, and indicator solution are introduced via pumps that go into the flow cell (e.g., using aliquots on the order of 100 μL-10 mL). The general procedure for the flow cell is to rinse the electrode with buffer, introduce the virus solution, then rinse with more buffer solution, and optionally filter. The indicator reagent is added to the QCM flow cell and act as a “stain” for the proteins on the virus surface, followed by a final rinse step. The indicator chemistry used at this stage of development is non-specific and produces a selective colorimetric response based exclusively on the selective binding of the virus by the MIP. The entire process for MIP analysis on QCM is buffer rinse>virus loading>rinse>indicator>rinse. All while recording frequency data in real time. Virus samples may consist of simple virus solutions and synthetic solutions that try to mimic the matrix and common biomolecule species from patient samples to test for interference at different concentrations of the virus.

After the MIPs have achieved the stated limit of detection in the simple systems they will be screened against patient samples with known COVID-19 diagnosis and ideally known viral loads by cross-testing the samples with polymerase chain reaction (PCR) techniques, which is currently the standard diagnosis technique for SARS-CoV-2 detection.

### Example 2. MIP QCM Sensor Synthesis

### General Methods:

### Virus Preparation:

SARS-CoV2 production: Local passage 2 (LP2) stocks of SARS-CoV-2/CANADA/VIDO 01/2020 were produced in Vero (Female green monkey kidney) E6 cells which were infected in a medium consisting of Dulbecco's Modified Eagle's medium supplemented with 1×non-essential amino acids (Gibco), 10 mM HEPES, 2% fetal bovine serum and cultured for 48 hrs. Media was harvested for determination of virus levels and for quantitation by qRT-PCR. In activation of virus was done by adding 1/10 volume of 10% (v/v) Triton X-100 (final concentration 1%)

SARS-CoV2 plaque assay: Virus levels were determined by a plaque forming unit assay. Vero E6 cells were infected with LP2 stocks 10 fold serially diluted in duplicate and after 1 h, the infecting medium was removed and monolayers were overlaid with growth media (MEM supplemented with 10 mM HEPES) containing 1.2% Avicel RC-591 (DuPont). After 48 h, cells were fixed in 10% formaldehyde, and stained using 0.5% (w/v) crystal violet and plaques were counted.

SARS-CoV2 RNA quantitation: Stocks of LP2 cell culture supernatant (140 μL) were collected and RNA was isolated using the QIAmp Viral RNA Mini kit as per manufacturer's instructions (Qiagen). Reverse transcription was carried out on 2 μL using Superscript IV Vilo master mix (Invitrogen). Quantitative PCR was carried out using 2 μL of cDNA in TaqMan Fast Master mix using primers and probes for the N gene (N₂ primers) designed by the United States center for disease control and prevention (IDT cat #10006606). The standard curve was generated using dilutions of positive control standards from CDC (IDT cat #10006625). The copy number was determined using the standard curve.

SARS-CoV2 purification: Virus was purified using ultracentrifugation through a sucrose cushion followed by iodixanol gradient centrifugation. Briefly 6 T-150 flasks were infected as above with SARS-CoV2 and the cell culture supernatant harvested and concentrated by centrifugation through a 30% sucrose cushion. The pellet was resuspended and then run on a 10-40% iodixanol gradient, and the fractions harvested and assessed for virus as above.

SARS-CoV2 inactivation: Virus was inactivated by crosslinking the genome with psoralen. For use outside the CL-3 facility the virus was inactivated using psoralen. Psoralen (Sigma) was added (fc=10 ug/ml) to purified virus fractions and irradiated with 3600 μJ of UV-A. To ensure virus inactivation, 10% of the fraction was placed on a well of a 6 well plate of Vero E6 cells and cultured for 6 days.

Cleaning Solid Support:

All glass slides and QCM resonators were cleaned by 5 min sonication in 95% EtOH and dried under nitrogen stream. Additionally, piranha solution was used for cleaning in some cases. 3:1 concentrated H₂SO₄:30% H₂O₂ heated to 60° C., soaked for 15 minutes, rinsed with DI water, and dried under a stream of N₂ or in an oven.

Microscopy System:

Data acquisition was performed on a Olympus spinning disk Confocal microscope with the following features:
Olympus IX-81 motorised microscope base
Yokagawa CSU 10 (#1) or CSU-X1 (#2) spinning disk confocal scan-head:
   Sedat Dichroic with reflections at 405/491/561/640 for standard 4-colour imaging
   CFP dichroic with reflections at 440/491/561/640 for 4-colour imaging using CFP.
Lenses: 10×/0.3 Phl, 20×/0.75 Dry (#1) 20×/0.85 Oil (#2), 40×/1.3 Oil, 60×/1.42 Oil
Illumination:
   X-Cite 120 from Lumen Dynamics for visualization with eyepieces
   LMM5 from Spectral Applied Research for laser merging.
   44 mW 405 nm pumped diode laser (for blue dyes, e.g. DAPI)
   40 mW 440 nm pumped diode laser (for CFP)
   50 mW 491 nm pumped diode laser (for green dyes, e.g. GFP, YFP, Alexa 488, FITC)
   50 mW 561 nm pumped diode laser (for red dyes, e.g. mCherry, Alexa 546, Cy3, TRITC)
   45 mW 642 nm pumped diode laser (for far-red dyes, e.g. Cy5, Alexa 633, Alexa 647)
Filter Cubes for use with eyepieces (BRIGHTLINE filter sets from Semrock):
Emission Filters for use during Confocal Imaging: The system is fitted with two interchangeable filter wheels, one for imaging standard 4 colour (Blue, Green, Red, Far-Red) “fixed” samples, and one optimised for imaging fluorophores commonly found in “live” cell experiments. Note that with these emission filters, there are no excitation filters necessary, as the lasers provide single wavelength excitation. Additionally, there is a choice of three dichroic mirrors housed in the confocal scan unit. Most commonly used, the Sedat dichroic is compatible with 4 colour fixed imaging. The second choice is a dichroic that maximises the emission from GFP (EGFP), but at the expense of separating its emission from other fluorophores. Last is a dichroic that is designed for separating CFP from YFP.
“Fixed” filter wheel (for use with “fixed” samples)
   Polariser (for DIC imaging)
   DAPI (460/50)
   GFP (515/30)
   Cy3 (595/50)
   Texas Red (620/60)
   Cy5 (690/50)
Imaging using a Hamamatsu EMCCD (C9100-13).
Acquisition using Perkin Elmer's Volocity
ASI MS-2000 motorised XY stage with a piezo Z insert that has 100 μm travel.

Step A: Preparation of the Virus Template:

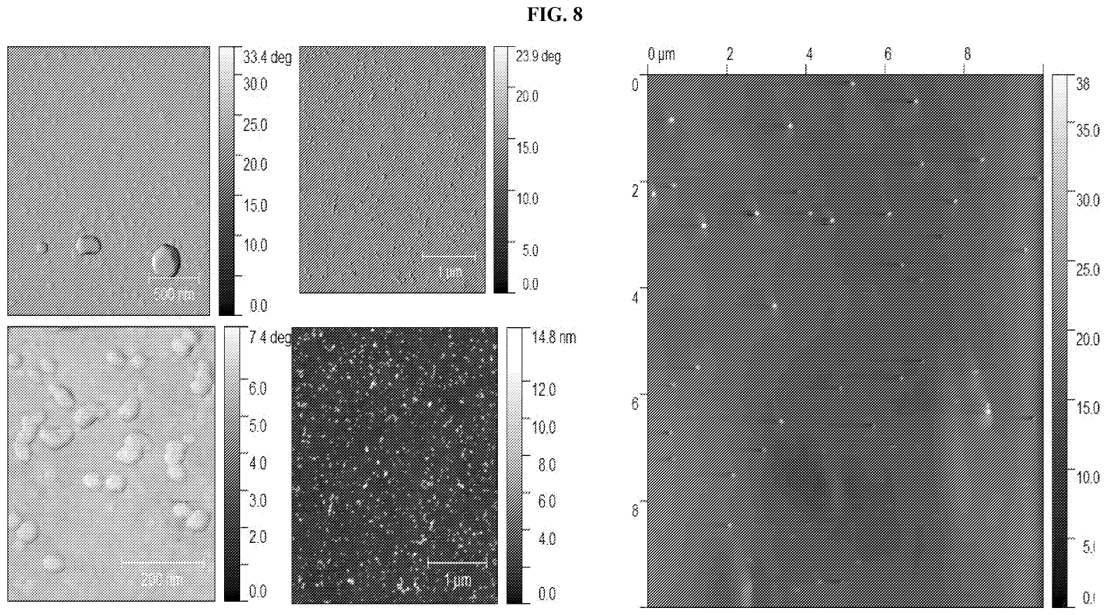

The concentrated virus stock solution (˜1E11-1E10 virus particles/mL) was diluted to a concentration 1E7 virus particles/mL with phosphate buffered saline (PBS) to prevent depositing of iodixanol from the virus solution and salt crystals from the buffer. Then pre-chilled (4° C.) glass slides were coated with the virus solution (600 μL for 25 mm×25 mm glass slide). The slides coated with the virus solution were refrigerated (4° C.) for 30-60 min to allow the virus to sediment. After sedimentation the glass slides were spin coated at 3,000 rpm for 3 seconds to remove excess liquid. The virus template was prepared immediately before use. The virus template was characterized via AFM to verify virus deposition density (see FIG. 8).

Alternatively, diodixanol be removed from the virus stock solution by using a 0.005 μm floating filter. To remove diodixanol from the virus stock solution, 50 μL of virus solution was placed on the filter floating in ˜250 mL of PBS and lightly stirred for 2 hours. This procedure allows close to the full concentration of virus stock solution to be used for the virus template making process

Step B: Preparation of MIP Thin Films

Preparation of spin coating solutions: for spin coating solutions poly(acrylic acid) (10 mg, 450,000 g/mol) was dissolved in 10 mL 95% EtOH. Then acrylamide (130 mg), methacrylic acid (104 μL), methylmethacrylate (64 uL), vinylpyridine (60.6 μL), and EGDMA (480 μL) were added. The resulting solution was then treating with initiator AIBN (10 mg) and the resulting solution degassed under N₂ for 15 minutes.

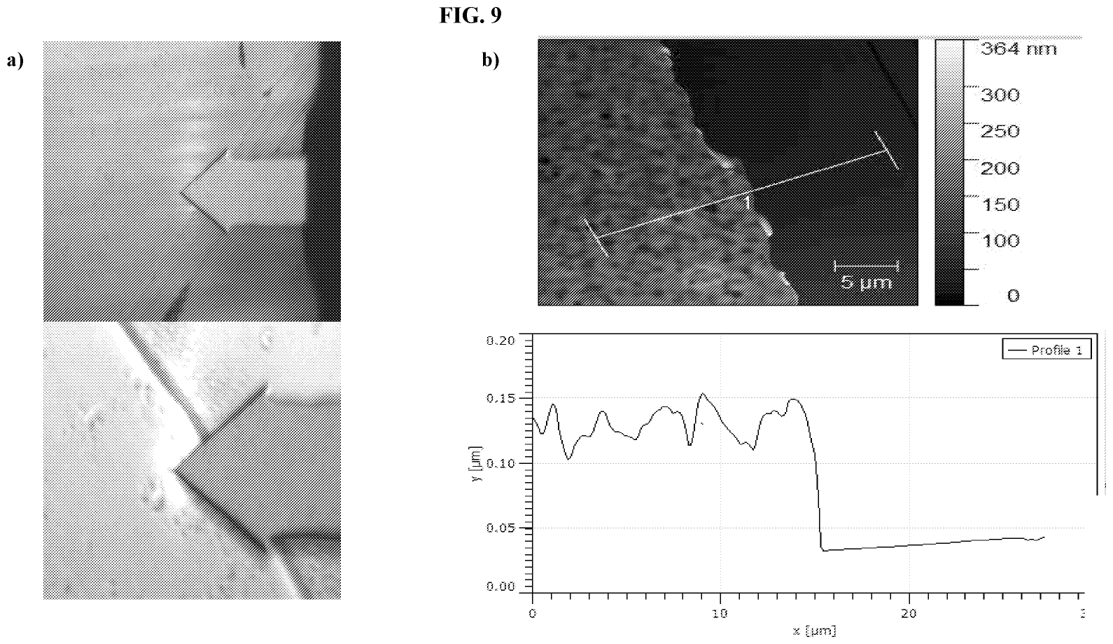

Spring Coating MIP film on gold QCM resonator or glass slide: The spin coater was programmed to spin at 2000 RPM for 5 seconds to allow for the addition of the reactive monomer solution and then increased to 8000 RPM for 30 seconds. For film formation 200 μL of the reactive monomer solution was added during the 2000 RPM spin cycle to coat a 1″ diameter QCM resonator or a 25 mm×25 mm glass slide to form thin films with a thickness of approximately 100-300 nm by AFM (See FIG. 9).

Step C: Imprinting Virus Template, Polymer Curing and Virus Elution

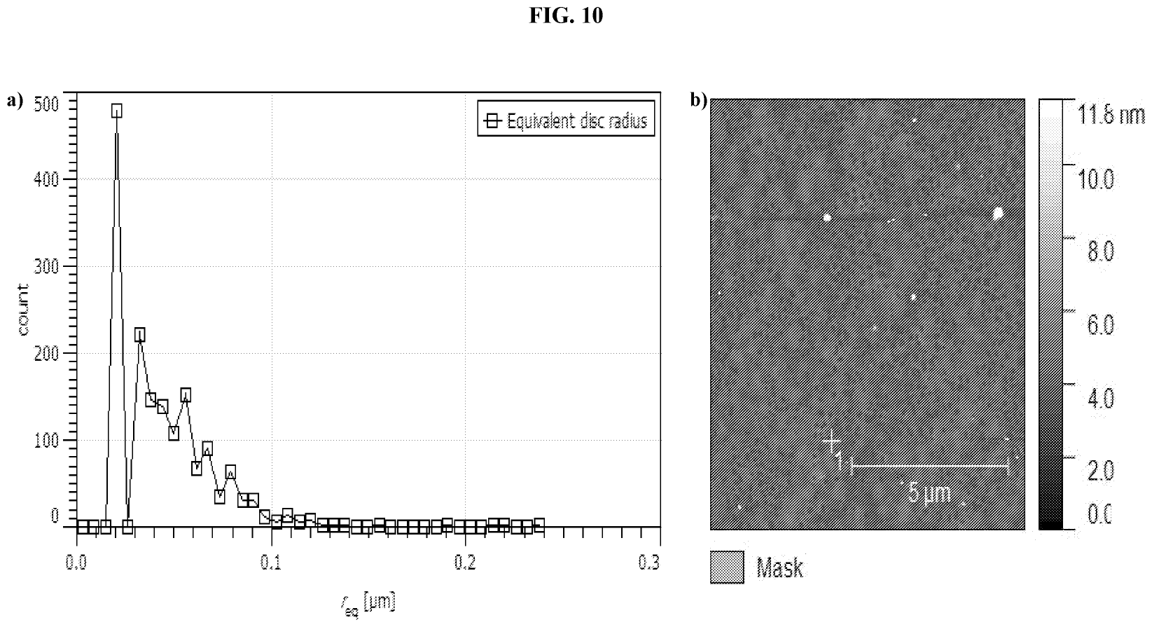

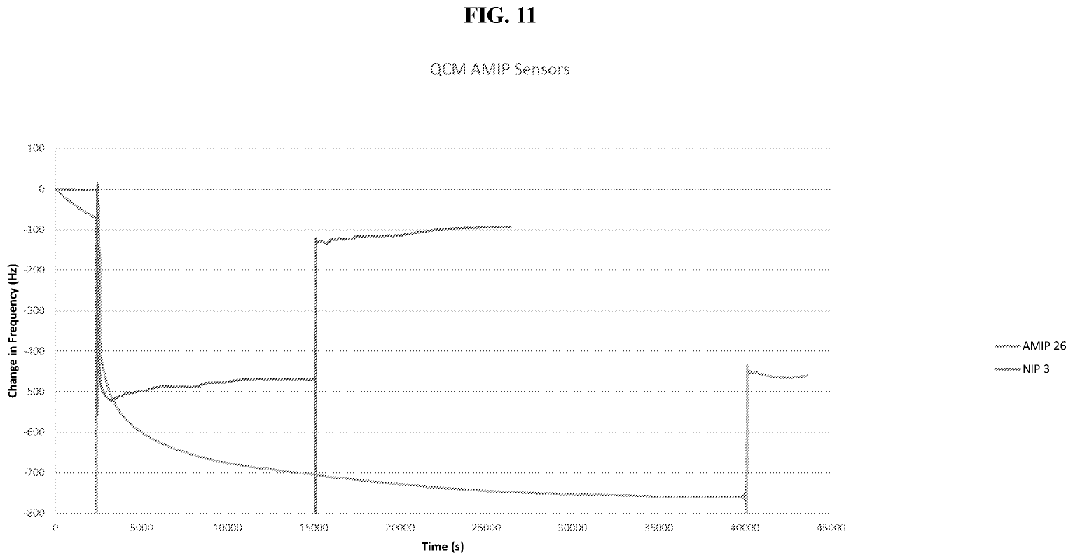

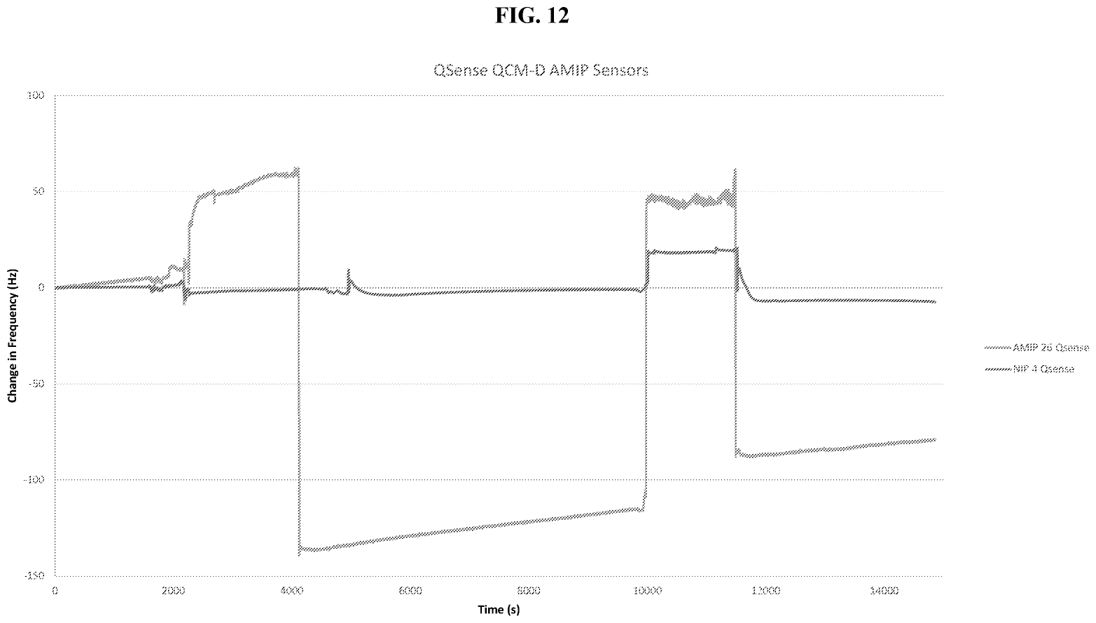

Immediately after spin coating the 25 mm×25 mm virus coated glass slide (the virus template) was pressed into the newly formed thin film for 30 seconds. The sandwiched slides were held together and placed under long wave UV light and allowed to cure overnight with the virus template glass slide up. After the curing step was complete, the sandwich was disassembled by peeling the virus template off the QCM resonator. The QCM resonator was then dipped in 1M HCl to denature the virus and rinsed with deionized water to remove/elute the virus. This was repeated 3× and then soaked in a water bath set to 50° C. with light stirring for 3 hours to fully remove the virus. Alternatively, the virus elution can be accomplished using 10% acetic acid has also been used in place of the 1M HCl. Imprinting was verified by AFM (FIG. 10) and adsorption performance measured by QCM (FIG. 11 and FIG. 12).

### Example 3. Evaluation of Surface Modifications of Gold QCM Resonator or Glass Slide for Spin Coating Step

Surface modification was evaluated to increase the bonding of the MIP thin film to the solid substrate (either gold QCM resonator or glass slide). For the gold surfaces the substrate was cleaned with piranha solution as described above and coated with dithiol alkanes to obtain self assembled monolayers (the SAMS interatected with the MIP thin film through thiol-ene click chemistry). The reaction was then diluted in 200 mL of absolute ethanol and allowed to react at room temperature for 72 hours. Afterwards the samples were rinsed with ethanol and dried under a stream of N₂ before progressing through the formulation described above.

For glass (and metal oxide surfaces in general) the surface was cleaned and hydroxylated with piranha solution as described above. The substrate was placed into a beaker with 1 mL of 3-(trimethoxysilyl)propyl methacrylate) and diluted with 200 mL of ethanol. The reaction was catalyzed with the addition of 6 mL of 10% acetic acid. After an exposure time of about 5 minutes the sample was rinsed with ethanol and dried under a stream of N₂. This procedure was used to attach vinyl groups to the surface of the glass plate.

### Example 4. QCM Procedure for the Preparation of Standard 5 MHz QCM (1″ Disks)

MIP quartz crystal sensor (such as, MIP coated on a 1″ 5 MHz quartz crystal) was placed in a flow cell with internal volume of ˜0.3 mL. The cell was closed and the frequency in air was measured to verify that it was close to the fundamental frequency of 5 MHz. The flow cell was then flushed with PBS buffer, the null adjusted, and allowed to stabilize for at least about 4 hours. After a stabilized baseline was achieved the flow cell was flushed with air (to maintain virus concentration) and 0.3 mL of virus was injected into the flow cell (virus concentration of 1E10-1E11), then the null adjusted. The signal was allowed to stabilize and the change in Hertz measured. The cell was flushed with ˜5 mL of PBS buffer and the null adjusted and the signal allowed to stabilize.

### Example 5. Fluorescence Based Colorimetric Detection/Pseudo ELISA Test

MIP disks from example 4 were activated by the 1M HCl treatment and DI water wash described above in Example 2.

Disks with active MIPS were incubated with cell culture supernatants from Vero E6 cells that were either uninfected or SARS-CoV-2 infected for 2 hrs at room temperature. Disks were briefly washed and labeled using Alexa Fluor 488 NHS ester (Thermo Fisher) by adding 100 uL of 0.1 MNaHCO₃ buffer containing 1/10 volume of Alexa488 NHS ester on the disk and incubating the disk for 1 hr at room temperature. Disks were washed, fixed using 4% paraformaldehyde for 15 min, washed again and then a coverslip was place on top using ProLong glass mounting media (Thermo Fisher). Samples were then imaged using an Olympus spinning disk Confocal microscope. The green channel was collected using the GFP filter. In multi-channel images the yellow channel was collected using the Cy3 filter and the red channel was collected with the Texas red filter. The lasers were on at the same time.

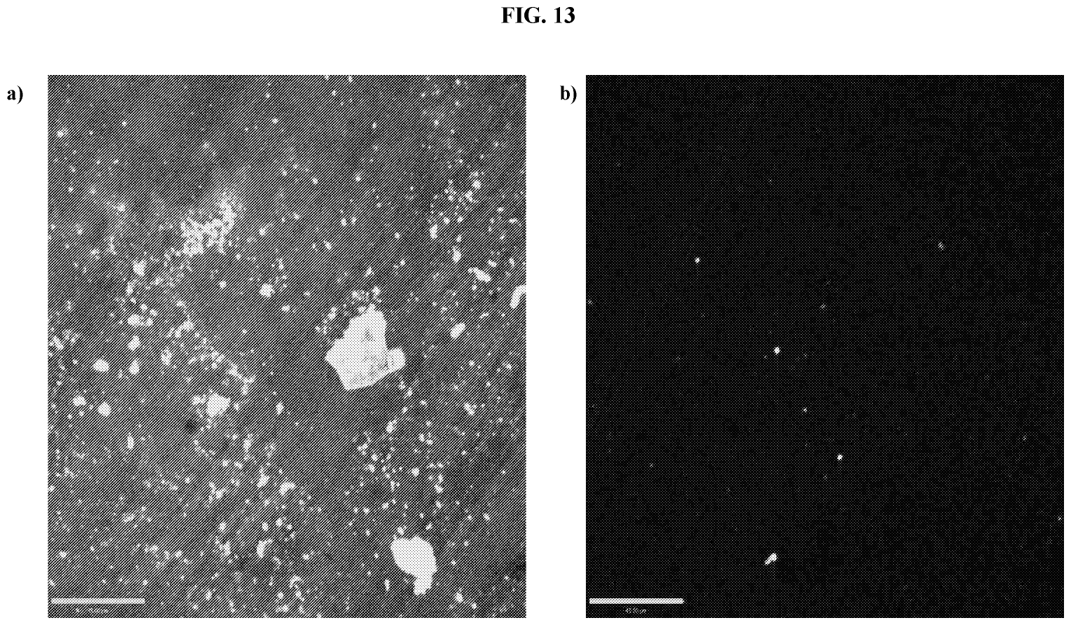

The MIP thin film showed measurable binding and detection of SARS-CoV-2 infected cells. As shown in FIG. 13, thin MIP films showed significantly more infected viruses bound to the MIP film (FIG. 13 a)), than uninfected cells bound to the MIP film (FIG. 13 b)).

### VIP (Virus Imprinted Polymer)+Chemical Labeling

a. Chemical Reaction Between Label Molecule and Adsorbed Virus
i. Microwell Assay

Technology platform: Independent molecular recognition and detection.

MIP is coated onto the bottom of a microwell plate, or a disc or other object inserted into the bottom of the micrwell plate. Patient sample is exposed then rinsed to remove non-binding material. A label molecule is added to bind to the virus (non-competitive) attached to the MIP. The label molecule essentially works as a stain and allows for detection of the virus through colorimetric, fluorescent changes or other suitable detection methods.

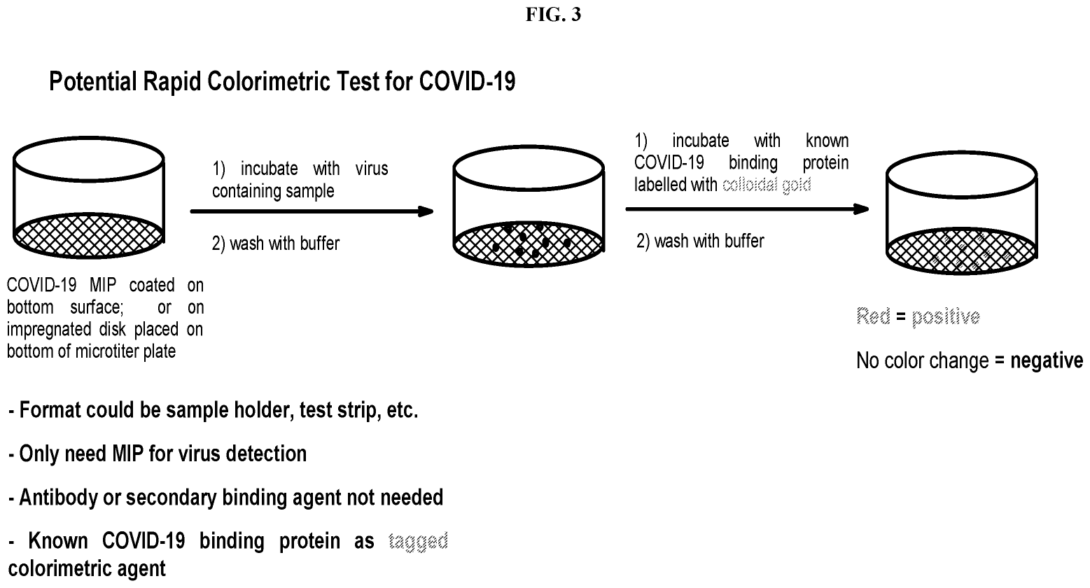

An exemplary microwell assay is depicted in FIG. 3. As shown in FIG. 3 SARS-CoV-2 selective MIPs are coated on the bottom surface of a microwell plate, or on an impregnated disk placed at the bottom of the plate. A SARS-CoV-2 containing sample is then exposed to the MIP coated plate and incubated. The plate is then washed with buffer, optionally filtered and incubated with a known SARS-CoV-2 binding protein labelled with colloidal gold, which provides a distinct red to orange color change in samples positive for SARS-CoV-2, and no color change in samples negative for SARS-CoV-2.

Exemplary microwell test kits of the present disclosure comprise a sample well coated with selective MIPs on the bottom surface, a positive control well, and a negative control well. The patient takes their sample (e.g., nasal swab) and mixes it with buffer solution within the sample well. Any viral particles adhere to the MIP coating. The patient will mix the sample for about 10 minutes to allow for full adsorption. Buffer is added to the two other wells. After the 10 minutes the microwells emptied by inversion and the sample well rinsed with buffer and optionally filtered. Next an indicator reagent is added to each well and allowed to react with the viral proteins and causes a color change. If the sample is positive, the MIP bound virus will react with the indicator reagent. If no virus is present, the indicator reagent will not react. The positive control well has a molecule coated on the bottom that reacts with the indicator reagent. The negative control well has no coating and thus no reaction. Next, all wells are rinsed with buffer and emptied by inversion. The three wells will give a color change for the positive well, no color change for the negative well, and potential color change in the sample well if the virus is detected.

ii. Lateral Flow Cartridge

Technology platform: Cartridge type molecular recognition with primary or secondary detection.

The lateral flow immunoassay is a colorimetric platform, which is normally used in determining a biomolecule or a chemical element in a solution with the aid of a color reagent. This method is widely applied to the analysis of solution samples in medical or industrial fields. It enables the detection of target molecules by the naked eye without any sophisticated instruments.

In exemplary lateral flow test kits of the present disclosure, the sample is taken and put in the sample well of the lateral flow test. Buffer is added to enhance the capillary action of the sample. The sample passes over a line of MIPs and if any viral particles are present, they bind to the MIPs. This binding event induces an observable colorimetric response. A control line verifies that the test is operating correctly.

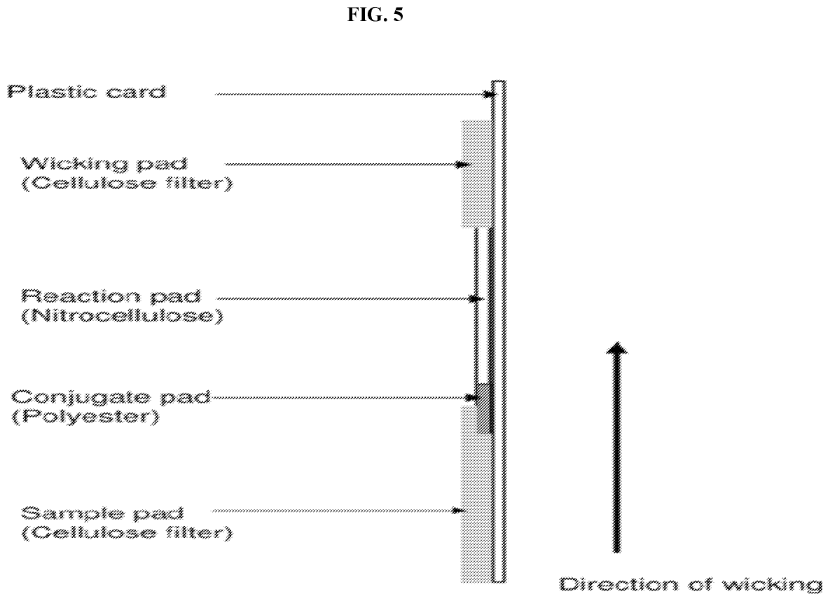

FIG. 5 shows the configuration of a typical lateral flow dipstick. The lateral flow dipstick (LFD) is composed of a sample pad, conjugate pad, reaction pad, wicking pad, and plastic card. For virus detection, MIPs are coated on the conjugation pad and the reaction pad. The biotinylated bovine serum albumin is doped onto the nitrocellulose membrane of the reaction pad. During the detection process, the surface color of the reaction pad is changed by adsorption of virus onto the MIPs and activation of a correlating molecular tag.

iii. Surface Contact Wipes with VIP Combination

Technology platform: Coupled molecular recognition and specific detection.

A wipe is used to check surfaces for viral contamination. The wipes are embedded with several different MIPs that are imprinted for different classes of viruses based on size and shape. Each MIP gives a different colorimetric response and can be used for testing unknown viruses.

iv. Diagnostic Mask

Technology platform: Coupled molecular recognition and specific detection.

Currently when patients come into a hospital setting with symptoms of a respiratory infection they are asked to put on a surgical mask to protect the other people at the hospital. Then they proceed to check-in at their doctor's appointment/ER visit, sit in a waiting room, get checked in by a nurse, all before finally seeing a doctor. To improve this processes MIPs for virus detection are incorporated inside the surgical mask (the side exposed to the patient's breath). As the patient breathes and coughs, the inside of the mask is moistened and virus particles are concentrated in the MIPs as the patient waits to see the doctor. At a certain limit of detection a color change indicates a viral infection, and thereby provides an effective and inexpensive diagnostic tool that can be employed before any expensive tests are done. With continuous wearing the mask can concentrate the virus ˜100× in 10 minutes and ˜1000× in 1 hour, a waiting period which is not an uncommon occurrence at many hospital settings. This allows for fast and passive diagnosis resulting in faster turnover for emergency rooms/urgent care settings.

v. Sticky Mat or Anti-Viral Coating

Technology platform: Coupled molecular recognition and specific detection.

Peoples' feet are constantly contacting the ground and are a source of virus spread. In extreme clean areas like an operating room in a hospital, a mat incorporating the MIPs of the present disclosure is used to “test” the feet/shoes of everyone that comes in. A color change in the mat is an indicator that the person/their clothes are also contaminated.

vi. Hospital Clothing

Technology platform: Coupled molecular recognition and specific detection.

MIPs as disclosed herein and an indicator can be incorporated into scrubs of other clothing used in a medical setting to show that the wash procedure was effective. A “negative” indication from the indicator shows no viral contamination.

vii. Air Handling/Sampling (could Include Multiple VIPs)

Technology platform: Coupled molecular recognition and specific detection.

Direct detection of viruses on an air handling filter or a continuous sampling from the air handling system that is bubbled through a reactor with MIP indicators for concentrating.

viii. Nasal Swab (could Include Multiple VIPs)

Technology platform: Coupled molecular recognition and specific detection.

Current sampling technique being used for respiratory infections with the MIP indicator embedded within the cotton swab

ix. Food Processing Coating (or Coating, Generally)

Technology platform: Coupled molecular recognition and specific detection.

Coating on food handling or processing equipment to check for viruses.

x. VIP “Flypaper” for Insect Sampling

Technology platform: Coupled molecular recognition and specific detection.

Detection in insects (Zika, dengue fever) fly tape with MIPs in the adhesive. For mosquitoes, test by setting up a feeding station, a thin membrane filled with a liquid that the insects will feed on. The mosquitoes will use the feeding station and release the virus into the apparatus. A MIP coating will adsorb the viruses released into the solution and cause a color change. Alternatively, a fly trap that consist of a bag with a one way entrance and liquid bait. Insects buildup in the Fly trap with the liquid being the media by which the virus is transported to the MIP coatings inducing the color change. The product can be used to study the spread of a disease (Zika), first monitoring sites by sampling insect populations.

xi. Hospital Spray for Visualizing Viral Contamination (Colorimetric Label Disappears Upon Disinfection)

Technology platform: Coupled molecular recognition and specific detection.

Suspended MIPs that can be sprayed. The MIPs react with virus and can be visualized.

xii. Sterilization Indicator

Technology platform: Coupled molecular recognition and specific detection.

Put in autoclave as a control.

xiii. Visualize Cell Infection (Lab Research Tool)

Technology platform: Coupled molecular recognition and specific detection.

Thin film of MIP with cells cultured on top. As the virus spreads from cell to cell the MIP will bind a fraction of them and the progression of the infection can be visualized via fluorescent microscopy.

xiv. Badge

Technology platform: Coupled molecular recognition and specific detection.

Badge on scrubs to indicate exposure limits for hospital workers. Similar to radiation badge

xv. Headband/Patch for Pediatric Use

Technology platform: Coupled molecular recognition and specific detection.

Headband or patch for continuous monitoring of sweat or drool. Patch with microneedles would allow for blood sampling as well.

xvi. Aquaculture Detector for Fish Virus

Technology platform: Coupled molecular recognition and specific detection.

Indicator for aquariums, fish virus.

xvii. Pet Chew Toy

Technology platform: Coupled molecular recognition and specific detection.

Most human viruses are contagious to domesticated animals like dogs and cats. A chew toy with a MIP indicator could be used to sample the pets saliva and be used in an extended exposure application.

xviii. Biological Fluid Test Kit (Urine, Fecal)

Technology platform: Coupled molecular recognition and specific detection.

GI sampling.

xix. Actively Monitoring Condensate from HVAC Systems

A detector (device or surface coated with MIPs of the present disclosure) is contacted with the condensate water of the HVAC system to provide continuous monitoring of the presence of the target virus in the HVAC system.

### VIP+Sensor-Based Detection

VIP Coated onto Sensor (Integrated Device)
i. Quartz Crystal Microbalance (QCM) Sensor

Technology platform: Independent molecular recognition and detection.

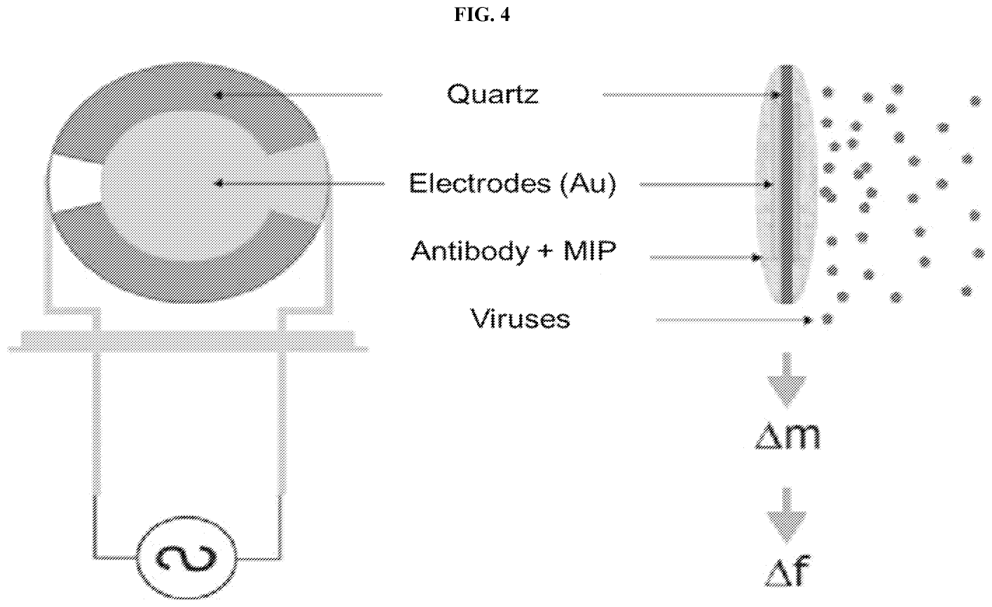

A QCM sensor is a kind of mass sensitive sensor. The basic material of the QCM sensor consists of quartz crystal, which is equipped with metal electrodes (e.g. gold). A sensor surface is modified with coating of MIPs, which is used to enable detection of viruses. An appropriate electronic circuit is necessary to make conversion of the measured virus quantity to an electrical signal. The basic working principles of the quartz crystal microbalance sensor is displayed in FIG. 4. Viruses that are present in the surrounding solution of a QCM sensor will interact with the sensor surface. In this interaction, viruses are bound to the MIP. The bound virus results in a mass change on the sensor surface. Consequently, the mass change on the sensor surface is converted to a frequency change. A QCM sensor is a useful tool for biomolecule detection and it has advantages such as high sensitivity, low response time, continuous operation, portable device, label-free, and real-time detection ability. QCM biosensor systems are composed of three components which are receptor, transducer, and signal monitoring system.

ii. SPR Coated Plate

Technology platform: Independent molecular recognition and detection.

Thin coatings of MIPs on surface of gold plated slide. When binding occurs the refractive index properties of the gold coated glass is altered and measurable. Quantitative

iii. Air Handling/Sampling

Technology platform: Coupled molecular recognition and specific detection.

Direct detection of viruses on an air handling filter or a continuous sampling from the air handling system that is bubbled through a reactor with MIP indicators for concentrating.

iv. Breathalyzer

Technology platform: Cartridge type molecular recognition with primary or secondary detection.

Most likely some type of electrochemical sensor for testing people's breath for virus particles

v. Electrochemical Biosensor

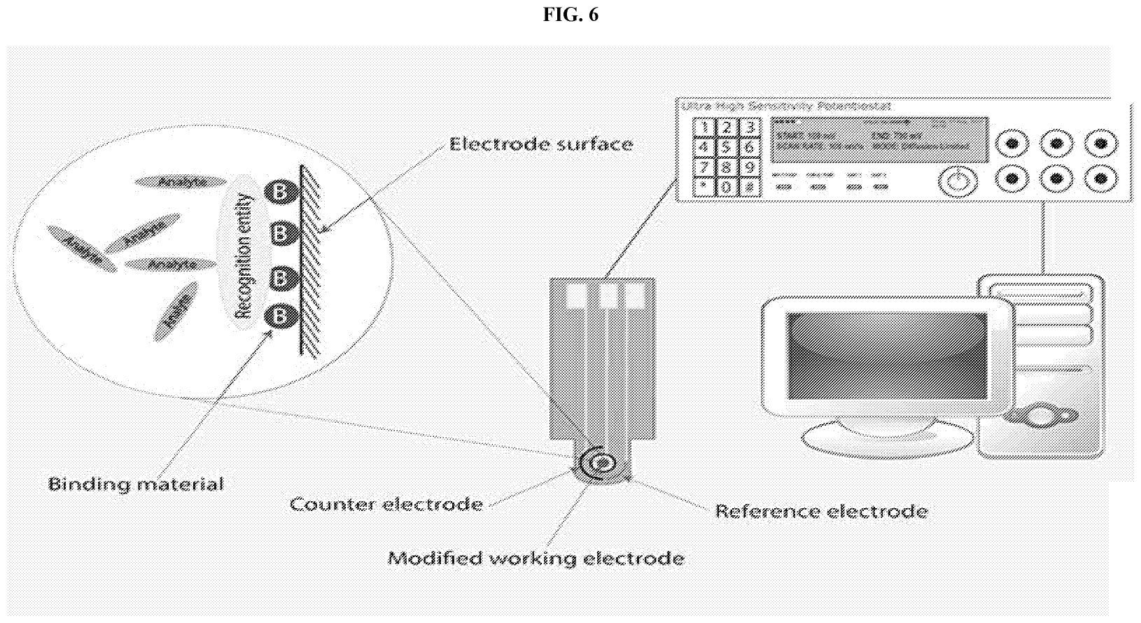

Electrochemical biosensors have electrodes which translate the chemical signal into an electrical signal. Electrochemical sensor can detect various biomolecules in the human body such as glucose, cholesterol, uric acid, lactate, DNA, hemoglobin, blood ketones, and viruses. Electrochemical biosensor typically requires a working electrode, a counter (or auxiliary) electrode and a reference electrode. The reference electrode is maintained at a distance from the site of the biological recognition element and analyte interaction to establish a known and stable potential. The working electrode acts as the transduction component when the interaction occurs whereas the counter electrode measures current and facilitates delivery of electrolytic solution to allow current transfer to the working electrode. The working and counter electrodes should be conductive and chemically stable and are mostly composed of carbon or inert metals like gold and platinum whilst the reference electrode is typically silver or silver chloride. A schematic diagram of an electrochemical biosensor is presented in FIG. 6.

Electrochemical Blood Test (Ex. Blood Glucose Test).

Technology platform: Cartridge type molecular recognition with primary or secondary detection

Blood borne diseases like HIV. Detection using an electrochemical response from virus binding to the MIP.

vi. Water Analysis

Technology platform: Cartridge type molecular recognition with primary or secondary detection. Indicator port for continuous monitoring of water supply, and/or indicator before and after filtration system for visual verification of water purity.

vii. AFM (Research Tool)

Technology platform: Independent molecular recognition and detection.

Coating on microcantilevers can be used to measure the binding strength of the MIP to a virus. Can study a single virus. Or different coatings on different cantalivers can be used for multiple analysis on a sample quickly.

viii. Magnetic Nano-Particles

Technology platform: Independent molecular recognition and detection.

There are several unique detection methods and device designs associated with magnetic nanoparticles. Magnetic nanoparticles also allow for the locational manipulation of the virus attached MIPs. Targeted infection. Magnetic immunoassay. Hall sensor.

ix. Optical Fibers

Technology platform: Cartridge type molecular recognition with primary or secondary detection. Surround optical-fiber immunoassay (SOFIA) is an ultrasensitive, in vitro diagnostic platform incorporating a surround optical-fiber assembly that captures fluorescence emissions from an entire sample. The technology's defining characteristics are its extremely high limit of detection, sensitivity, and dynamic range. SOFIA's sensitivity is measured at the attogram level (10-18 g), making it about one billion times more sensitive than conventional diagnostic techniques. Based on its enhanced dynamic range, SOFIA is able to discriminate levels of analyte in a sample over 10 orders of magnitude, facilitating accurate titering

x. microarray

Technology platform: Cartridge type molecular recognition with primary or secondary detection. Microfluidics and micro arrays are ways to screen large library's of ligands, often DNA or proteins, with a small amount of sample. Microchannel in PDMS are used to transport small amounts of liquid/sample for fast kinetics, reactions, and multiple testing sites. This lab on a chip concept is well developed in the literature.

xi. RFID Based Biosensor

The present disclosure also provides, in part radio-frequency identification (RFID) biosensors comprising a MIP film of the present disclosure for use in detecting target virus(es).

In some embodiments, the RFID device comprises a MIP film and an antenna. Detection in such devices may be based on the binding of the target virus to the MIP film which triggers a detectable change in the resonance frequency of the antenna. For example, in some embodiments, the RFID device operates like an on-off switch, wherein binding of the virus changes the resonance frequency of the RFID antenna, such that the RFID is not able to respond to a RFID field generator in sufficient proximity. In some embodiments, the MIP film is deposited on the resonant antenna.

In some embodiments, the biosensor comprises a) a first layer deposited on top of a RFID device (such as an antenna), comprising a conductive polymer (such as PEDOT:PSS), and b) a second layer deposited on top of the first layer comprising a MIP of the present disclosure (e.g., a MIP film as disclosed herein).

In some embodiments, the RFID devices comprise an antenna, a MIP film and data-carrying means which is electronically programmable with unique information (e.g., microchip, integrated circuit and the like). In some embodiments of such devices, the data encoded in the data-carrying means can be decoded by a reader and transmitted to a host computer (information management system) for processing.

In some embodiments, the RFID device comprises a microchip with input/output (I/O) ports which are configured to pass through a MIP of the present disclosure. In some embodiments, the binding of the MIP of the present disclosure can cause the device to become conductive. In other embodiments, the binding of the MIP of the present disclosure can cause the device to become non-conductive. Accordingly, in the presence and absence of the virus, the conductivity of the device will be different. In some embodiments, the chip provides multiple free ports to allow for multiple MIPs for different viruses to be implemented in the same RFID sensor. In some embodiments, the RFID device can provide a quantitive value (e.g., based on resistance or capacitance changes upon virus binding), enabling the concentration of target virus(es) to be remotely measured.

In some embodiments, the RFID-based biosensor is a LC or LCR-based biosensor which comprises a suitable resonating inductor-capacitor (LC) or inductor-capacitor-resistor (LCR) circuit known in the art, and described for example in Sensors 2017, 17, 2289. In such embodiments, the capacitor may be covered with a MIP film of the present disclosure and the chemical binding of the target virus(es) to the MIP layer changes the electrical properties of the LCR device (e.g., permittivity, impedance, resonance frequency, and the like) which can be detected e.g., by a remote reader.

### VIP Adsorbent

a. Purification
i. Water Treatment

MIPs can be used as a virus adsorbent for continuously monitoring and sequestering virus from water samples.

ii. Dialyzer for Therapeutic Use in Infected Patients

Technology Platform: Solid Phase Extraction

MIP coated tubing, high surface area device etc. adsorbs virus from blood and purifies it before returning the blood to the patient. Will help reduce viral particles in patients with critical infections, by reducing free circulating particles. Can be used for removal of viruses (hepatitis) from donated blood.

iii. Breast Milk (or Other) Purification

Technology platform: Coupled molecular recognition and specific detection.

Indicator to see if virus from sick mother is being passed through breast milk to the baby.

iv. Stick Mat or Coating for Hospital Use (Sequestervirus)

Technology platform: Coupled molecular recognition and specific detection.

Peoples' feet are constantly contacting the ground and could be a source of virus spread. In extreme clean areas like an operating room in a hospital, a mat including MIPs of the present disclosure are used to “test” the feet/shoes of everyone that comes in. The detection of virus particles on the feet are an indicator that the rest of the person/their clothes are also contaminated.

v. Protein Template in Protein Synthesis

Technology Platform: Solid Phase Extraction

E. coli is often used for the synthesis of viral proteins in vaccine production. A MIP cavity is used to help promote folding of the protein into the correct shape after synthesis by the E. coli.

vi. Sampling (Animal Feed/Water. Public Facilities)

Technology platform: Coupled molecular recognition and specific detection.

Livestock often have communal resources such as a feeding trough or watering dish. These communal resources are used as a sampling point for viral infections.

vii. Viral Harvesting

Technology Platform: Solid Phase Extraction

MIPs are used as a virus adsorbent to concentrate the virus for sampling and analysis with other techniques.

viii. Antigen Harvesting

Technology Platform: Solid Phase Extraction

When antigens are grown for vaccine use they need to be harvested and purified. Current techniques use column chromatography and special protein tags to promote selective binding. MIPs as disclosed herein are the purification media in this application.

ix. HEPA Filters with MIPs to Get Virus Adsorption.

Virus particles are generally too small to be filtered by HEPA filters; MIPs of the present disclosure are added for virus adsorption.

1. A molecularly imprinted polymer (MIP) comprising a crosslinked copolymer having a plurality of complexing cavities which selectively bind one or more viruses of a target viral genus, wherein the copolymer is prepared by polymerizing a mixture comprising:
(a) one or more binding monomers complexed to a template having a size, shape and morphology substantially similar to the viruses of the target viral genus;
(b) optionally one or more non-crosslinking monomers; and
(c) one or more crosslinking monomers;
then removing the template from the copolymer, thereby providing said plurality of complexing cavities.

2. The MIP of claim 1, wherein the target viral genus is Betacoronavirus.

3. The MIP of claim 1, wherein the complexing cavities are selective for SARS-CoV-2.

4. The MIP of claim 1, wherein the template is an inactivated form of the target virus.

5. The MIP of claim 4, wherein the template is an inactivated virus of the genus Betacoronavirus.

6. The MIP of claim 4, wherein the template is an inactivated SARS-CoV-2 virus.

7. The MIP of claim 1, wherein the one or more binding monomers are selected from the group consisting of acrylic acid, methacrylic acid, acrylamide, methacrylamide, vinylpyridine, N-alkylacrylamides, N-isopropylacrylamide, hydroxyethylmethacrylate, and combinations thereof.

8. The MIP of claim 1, wherein the one or more non-crosslinking monomers are selected from the group consisting of alkylmethacrylates, methylinethacrylate, ethylmethacrylate, and combinations thereof.

9. The MIP of claim 1, wherein the one or more crosslinking monomers are selected from the group consisting of ethylene glycol dimethacrylate (EGDMA), N, N′-methylenebisacrylamide, N,N′-(1,2-dihydroxyethylene)bisacrylamide, and combinations thereof.

10. The MIP of claim 1, wherein the mixture further comprises a viscosity modifier.

11. The MIP of claim 10, wherein the viscosity modifier is a polymer selected from the group consisting of poly(acrylic acid), poly(ethylene oxide), poly(ethyleneimine), poly(propylene oxide), copolymers of ethylene oxide and propylene oxide, block copolymers of polyethylene oxide (PEO) and polypropylene oxide (PPO), triblock PEO-PPO-PEO polymers, and combinations thereof.

12. The MIP of claim 11, wherein the viscosity modifier has a molecular weight (Mw) ranging from about 50,000 to about 500,000 g/mol.

13. The MIP of claim 1, wherein the mixture further comprises a photoinitiator.

14. The MIP of claim 13, wherein the photoinitiator is activated at a non-germicidal wavelength.

15. The MIP of claim 13, wherein the photoinitiator is selected from the group consisting of azoisobutyronitrile (AIBN), diphenyl(2,4,6-trimethylbenzoyl)phosphine oxide (TPO), 1-phenyl 1,2-propanedione (PPD), camphorquinone (CQ), and phenylbis(2,4,6-trimethylbenzoyl)phosphine oxide, and combinations thereof.

16. The MIP of claim 1, wherein the mixture comprises about 40% to about 70% (v/v) crosslinking monomer, about 30% to about 50% (v/v) binding monomer, and about 0.5% (v/v) to about 1.5% (v/v) photoinitiator.

17. The MIP of any of claims 2-16, wherein the sensitivity and selectivity of the MIP is at least 70%.

18. The MIP of any of claims 2-16, wherein the sensitivity and selectivity of the MIP is at least 90%.

19. A MIP array comprising two or more MIPs according to any of claims 1-18, wherein each MIP comprises a crosslinked copolymer having a plurality of complexing cavities which selectively bind a target virus, wherein the target virus of each MIP is different.

20. The MIP array of claim 19, wherein each crosslinked copolymer is prepared by polymerizing a mixture comprising:
(a) one or more binding monomers complexed to an inactivated form of the target virus for that MIP; and
(b) one or more crosslinking monomers to provide a copolymer;
(c) removing the inactivated virus from the copolymer,
thereby providing said plurality of complexing cavities for that MIP.

21. The MIP array of claim 19, wherein the chemical composition of each MIP is the same.

22. The MIP array of claim 19, wherein the chemical composition of at least one MIP is different from the chemical composition of one other MIP.

23. A MIP array comprising two or more MIPs of any of claims 1-18.

24. The MIP array of claim 23, wherein the chemical composition of each MIP is the same.

25. The MIP array of claim 23, wherein the chemical composition of at least one MIP is different from the chemical composition of one other MIP.

26. The MIP array of any of claims 19-25, wherein at least one MIP of the MIP array binds a virus of the genus Betacoronavirus.

27. The MIP array of any of claims 19-25, wherein at least one MIP of the MIP array binds SARS-CoV-2.

28. The MIP array of claim 26, wherein the sensitivity and selectivity of the at least one MIP that binds a virus of the genus Betacoronavirus is at least 70%.

29. The MIP array of claim 27, wherein the sensitivity and selectivity of the at least one MIP that binds SARS-CoV-2 is at least 90%.

30. A microwell comprising a MIP of any of claims 1-18.

31. A cartridge comprising a container filled with a MIP of any of claims 1-18.

32. A quartz crystal microbalance resonator coated with at least one MIP of any of claims 1-18.

33. An article comprising a MIP of any of claims 1-18, and a labeling composition comprising a label, wherein the label binds to any target virus bound to the MIP.

34. An article comprising a MIP of any of claims 1-18 and further comprising a labeling composition comprising a label, wherein the label binds to any target virus bound to the MIP, then removing the virus from the copolymer, thereby providing said plurality of complexing cavities.

35. The article of claims 33 or 34, in the form of a fabric, face mask, swab, coating, badge, thin film, bead, fiber or sprayable liquid, which when contacted with the target virus or virus of a target viral genus, binds the labeling compound to any target virus or virus of a target genus bound to the MIP.

36. The article of any of claims 33-35, wherein the labeled target virus or labeled virus of a target genus bound to the MIP is detectable colorimetrically, by fluorescence, or magnetically.

37. A method of detecting a target virus or a target viral genus comprising:
(a) contacting a sample with a MIP of any of claims 1-18, thereby binding at least a portion of the target viral genus present in the sample to the MIP; and
(b) reacting a label molecule with the bound viruses;
wherein the label molecule can be detected to show the presence of the bound virus.

38. A method of detecting a target virus or a target viral genus comprising:
(a) contacting a sample with an array or article of any of claims 19-36, thereby binding at least a portion of the target viruses in the sample to one or more of the MIPs; and
(b) reacting a label molecule with the bound viruses;
wherein the label molecule can be detected to show the presence of the bound target virus.

39. A sensor for detecting a virus, coated with a MIP or MIP array of any of claims 1-29.

40. The sensor of claim 39, selected from a quartz crystal electrode, SPR plate, electrochemical sensor, magnetic particle, or optical fiber.

41. A method of detecting a target virus with a sensor of claims 39 or 40, wherein the sensor is exposed to a sample, whereby target virus in the sample binds to the MIP or MIP array coating, and the amount of target virus in the sample correlates to a change in sensor response to said binding of the target virus to the MIP or MIP array.

42. A method of removing one or more target viruses from a medium, comprising contacting the fluid with a MIP or MIP array of any of claims 1-29, whereby at least a portion of any target virus in the fluid binds to the MIP or MIP array.

43. The method of claim 42, wherein the medium is water.

44. The method of claim 42, wherein the medium is air.

45. The method of any of claims 42-44, wherein the MIP or MIP array is in the form of a container filled with the MIP or MIP array.

46. The method of any of claims 42-45, further comprising quantifying the amount of bound target virus.
